(19) United States
(12) Patent Application Publication
(10) Pub. No.: US 2026/0241087 A1
(43) Pub. Date: Aug. 20, 2026

(54) FLUID REACTOR AND FLUID REACTOR COMPONENT MANUFACTURING

(71) Applicant: CVD Equipment Corporation, Central Islip, NY (US)

(72) Inventors: Karlheinz Strobl, Mt. Sinai, NY (US); Sandra Gainey, Greenville, SC (US); Matthew Kesich, Brewster, NY (US); Jeffrey Brogan, Stony Brook, NY (US); Ajay Kumar, Bayport, NY (US)

(21) Appl. No.: 17/640,409

(22) PCT Filed: Sep. 4, 2020
(86) PCT No.: PCT/US2020/049466

§ 371 Date: Mar. 4, 2022

Related U.S. Application Data
(60) Provisional application No. 62/896,310, filed on Sep. 5, 2019.

(51) Int. Cl. A61M 1/16; B01D 61/36; B82Y 30/00; C02F 1/44

(52) U.S. Cl. CPC A61M 1/1698; B01D 61/364; B01D 61/366; C02F 1/447; B82Y 30/00

(57) ABSTRACT

A fluid reactor unit includes a fluid reactor housing and a reactor core. A reactor core includes at least one reactor core assembly made from reactor core assembly parts. Reactor core assembly parts are either reactor core components or reactor core accessories. A reactor core component includes a reactor core frame and at least one reactor core element having multiple fluid channel perforations that are surrounded by an open-pore cellular network material having a bi-continuous tortuous phase. Reactor core accessories can include, but are not limited to, heat exchangers, electrically power heaters, and endplates.

### TECHNICAL FIELD

This disclosure is directed to methods and systems used to build fluid reactors and compatible fluid reactor components, where the fluid reactors include a fluid reactor unit housing containing a reactor core that generally incorporates at least one free standing reactor core element having multiple fluid channel perforations that are surrounded by an open-pore cellular network material having a bi-continuous tortuous phase structure, and specifically incorporates at least one reactor core element including carbon-infiltrated vertically aligned carbon nanotubes.

### BACKGROUND

Fluid reactor units typically include a sealed fluid reactor unit housing that encloses a reactor core that includes at least one reactor core element. The fluid reactor units have, at a minimum, a primary fluid input port and a primary fluid output port. In addition, they often have at least one or two secondary fluid ports, with the secondary ports being either an input or output port. Many fluid reactor units additionally have auxiliary ports for various purposes, including: venting; defoaming; removing bubbles; sampling blood; inserting saline, a drug, or other materials; sensing temperature, flow rate, and/or pressure; sensing levels of oxygenation, CO₂, pH, salinity, etc. The fluid reactor units may also include auxiliary filters (particle filters, particle agglomeration filters, arterial filters, etc.) positioned in-line with the entrance and/or exit ports and/or built into the reactor core. The reactor cores contained in these fluid reactor units are most commonly designed as either a filter module, a spiral wound module, or a hollow fiber module.

Fluid reactor units incorporating a reactor core in the form of a filter module are typically two port devices with at least one component of the primary input fluid getting preferentially trapped inside the reactor core, i.e., inside the filter module. Therefore, fluid reactor units with these filter module reactor cores have a continuously decaying flow performance behavior as the filter media gets loaded up with an accompanying increasing pressure drop behavior.

Fluid reactor units that house reactor cores including spiral wound modules or hollow fiber modules are cross-flow devices where a primary input fluid flows primarily parallel to an active membrane surface. These fluid reactor units are, at a minimum, three-port devices with the third port being a secondary fluid output or input port which allows continuous removal or addition of a secondary fluid. For example, where a fluid reactor unit operates as a filter, a primary input fluid enters the device and is separated into 1) a concentrate, which exits the device as the primary output fluid, and 2) a permeate, which is continuously removed from the device as the secondary fluid. The additional continuous removal operation of the primary output fluid enables a much more steady and continuous filtering operation as compared to the operation of fluid reactor units having dual port filter module type reactor cores, since the buildup on the active membrane surfaces is stabilized (after an initial seasoning period) by the cross-flow fluid operation mode.

Fluid reactor units incorporating a reactor core in the form of a hollow fiber module, i.e., a bundle of many hollow fibers having a porous sidewall, are typically three or four port devices. A secondary fluid port provides a secondary input or output fluid to the reactor core and specifically to each reactor core element making up the reactor core. Such four port fluid reactor units may be used, for example, for blood oxygenation where the fluid reactor unit is used as an extracorporeal membrane oxygenator during a cardiopulmonary bypass surgery.

A fluid reactor may incorporate a fluid reactor unit fluid flow controller with a sensor and a control box that receives a demand signal. When the control box controls the fluid reactor unit fluid flow controller to minimize the difference between the present value and a set value of the sensor, the fluid reactors may be referred to as a dynamically adjusting fluid reactors.

Fluid reactors with spiral wound and hollow fiber separators may also be used for reverse osmosis water desalination and many other separator applications including liquid degassing, liquid gasification, and dialysis.

Despite the existence of these various types of fluid reactors, there remains room for improvement in the art of fluid reactors, especially with respect to structural configurations, materials of construction, and improving functionality, particularly in the field of blood treatment (e.g., oxygenation).

### SUMMARY

The present disclosure is directed to the manufacture and design of fluid reactors incorporating reactor cores that include at least one reactor core assembly having at least one reactor core component. The reactor core component may include at least one reactor subcomponent, where such reactor core subcomponent incorporates at least one freestanding reactor core element having multiple fluid channel perforations that are surrounded by an open-pore cellular network material having a bi-continuous tortuous phase structure, and/or specifically, incorporates at least one carbon-infiltrated vertically aligned carbon nanotube (c-VACNT) reactor core element.

A reactor core assembly in accordance with the present disclosure includes at least one reactor core assembly part. In some cases, the reactor core assembly part incorporates at least one reactor core element, and such reactor core assembly parts are sometimes referred to herein as reactor core components. In other cases, the reactor core assembly part does not incorporate any reactor core elements, and such reactor core assembly parts are sometimes referred to herein as reactor core accessories. This disclosure is also directed to various methods and systems for building reactor core assemblies that are suitable for a given application. For particular applications, by building performance-enhancing reactor core components, performance enhancing reactor core subcomponents, or reactor core accessories, the usability and/or performance range of a fluid reactor incorporating such reactor core components, subcomponents or accessories may be augmented.

In one aspect of the present disclosure, a reactor core component is provided and includes a reactor core frame having a first window, a second window, and defining a secondary fluid cavity. The reactor core component further includes at least one reactor core element having a primary fluid input surface, a primary fluid output surface, and a sidewall. The at least one reactor core element is secured to the reactor core frame via one or more fluid tight seals. At least one side of at least one of the first or second windows is larger in width or length than the corresponding width or length of the at least one reactor core element. When secured to the reactor core frame, at least a portion of the primary fluid input surface of the at least one reactor core element is exposed through the first window, at least a portion of the primary fluid output surface is exposed through the second window, and at least a portion of the reactor core element sidewall is positioned within the secondary fluid cavity. The primary fluid input surface of the at least one reactor core element includes a plurality of fluid channels extending through the at least one reactor core element to the primary fluid output surface. Each channel of the plurality of fluid channels is surrounded by an open-pore cellular network material having a bi-continuous tortuous phase structure, and defines a sidewall that allows a secondary fluid to pass through the sidewall of the channel while restricting the flow of at least one component of a primary fluid through the sidewall of the channel.

In embodiments, the at least one reactor core element may have a width smaller than its length. In embodiments, the at least one reactor core element may have a width to length ratio from 1:3 to 1:12.

In embodiments, the reactor core frame includes a first frame piece assembled with a second frame piece to form the reactor core frame, with the first frame piece defining the first window and the second frame piece defining the second window. In embodiments, turbulence generators may span from the first frame piece to the second frame piece in the secondary fluid cavity.

In embodiments, the reactor core frame may include a primary fluid input manifold sheet, an input frame sheet, an output frame sheet, a primary fluid output manifold sheet, and a pair of spacers between the input frame sheet and the output frame sheet.

In embodiments, the at least one reactor core element may be secured to the reactor core frame via a UV-curable glue.

In embodiments, the at least one reactor core element may be a reactor core stack including a plurality of reactor core elements. In such embodiments, two or more of the reactor core elements of the plurality of reactor core elements may be connected in parallel. In other such embodiments, two or more of the reactor core elements of the plurality of reactor core elements may be connected in series.

In embodiments, at least a portion of the open-pore cellular network material having a bi-continuous tortuous phase structure may be coated. In embodiments, at least a portion of the open-pore cellular network material having a bi-continuous tortuous phase structure is coated with a hydrophobic coating. In embodiments, the hydrophobic coating may be a hydrophobic polymer. In embodiments, the hydrophobic polymer may be a fluorocarbon material.

In another aspect of the present disclosure, a reactor core component is provided and includes a reactor core frame including a first frame piece assembled with a second frame piece, the reactor core frame having a plurality of pairs of windows, where each pair of windows of the plurality of pairs of windows is defined by a first window in the first frame piece, and a second window in the second frame piece. The reactor core frame also defines a secondary fluid cavity. The reactor core component also includes a plurality of reactor core stacks each having a primary fluid input surface, a primary fluid output surface, and a sidewall. Each reactor core stack of the plurality of reactor core stacks is secured to the reactor core frame between a respective pair of windows of the plurality of pairs of windows via one or more fluid tight seals. At least one side of at least one of the first or second windows of each pair of windows of the plurality of pairs of windows is larger in width or length than the corresponding width or length of the respective reactor core stack secured therein. For each reactor core stack of the plurality of reactor core stacks, at least a portion of the primary fluid input surface is exposed through the first window, at least a portion of the primary fluid output surface is exposed through the second window, and at least a portion of the reactor core stack sidewall is positioned within the secondary fluid cavity. The primary fluid input surface of the reactor core stack includes a plurality of fluid channels extending through the reactor core stack to the primary fluid output surface. Each channel of the plurality of fluid channels is surrounded by an open-pore cellular network material having a bi-continuous tortuous phase structure, and defines a sidewall that allows a secondary fluid to pass through the sidewall of the channel while restricting the flow of at least one component of a primary fluid through the sidewall of the channel.

In embodiments, at least one reactor core stack of the plurality of reactor core stacks includes two or more reactor core elements. In such embodiments, the two or more of the reactor core elements may be connected in parallel. In other such embodiments, the two or more of the reactor core elements may be connected in series.

In another aspect of the present disclosure, a reactor core assembly is provided and includes a first reactor core component in accordance with any of the aspects described above, and at least one of i) a second reactor core component in accordance with any of the aspects described above or ii) a reactor core accessory selected from a heat exchanger or a sensor.

In another aspect of the present disclosure, a fluid reactor unit is provided and includes a reactor core component in accordance with any of the aspects described above sealed within a fluid reactor unit housing including a first endplate and a second endplate. The housing includes a primary fluid input port, a primary fluid output port, and at least one of a secondary fluid input port or a secondary fluid output port.

In another aspect of the present disclosure, a fluid reactor unit is provided and includes at least one reactor core component in accordance any of the aspects described above sealed within a housing including a lid and a container. The housing has and a primary fluid input port, a primary fluid output port, and at least one of a secondary fluid input port or a secondary fluid output port.

In another aspect of the present disclosure, a fluid reactor unit is provided and includes a first plurality of reactor core assembly parts, the first plurality of reactor core assembly parts including at least one of a reactor core component in accordance with any of the aspects described above and optionally a reactor core accessory. The fluid reactor unit further includes a second plurality of reactor core assembly parts, the second plurality of reactor core assembly parts including at least one of i) a reactor core component in accordance with any of the aspects described above or ii) a reactor core accessory. The first and second pluralities of reactor core assembly parts are sealed within a housing including a lid and a container. The housing has a primary fluid input port, a primary fluid output port, and at least one of a secondary fluid input port or a secondary fluid output port.

In another aspect of the present disclosure, a fluid reactor unit is provided and includes a plurality of reactor core assembly parts sealed within a housing. The plurality of reactor core assembly parts includes at least two reactor core components in accordance with any of the aspects described above. The at least two reactor core components are positioned adjacent to one another such that a window of one of the at least two reactor core components is serially connected with a window of another of the at least two reactor core components.

In another aspect of the present disclosure a method of making a fluid reactor for compositionally changing a primary input fluid into a primary output fluid with at least one secondary fluid is provided and includes securing a reactor core component within a fluid tight fluid reactor housing. The reactor core component is prepared by loading a reactor core element into a window of a reactor core frame and bonding the reactor core element to the window. The reactor core frame has a secondary fluid cavity, and the reactor core element has a sidewall and a plurality of fluid channels surrounded by an open-pore cellular network material having a bi-continuous tortuous phase structure that is configured to be transmissive to a secondary fluid. At least one side of the window is larger in width and length than the reactor core element. The reactor core element is bonded to the window such that at least a portion of the reactor core element sidewall is not sealed.

In embodiments, loading a reactor core element into a window of a reactor core frame may include loading a reactor core stack into a window of a reactor core frame. In embodiments, the reactor core frame may include a plurality of windows, and the method may include loading each reactor core element of a plurality of reactor core elements into a respective window of the plurality of windows. In such embodiments, loading each reactor core element of a plurality of reactor core elements into a respective window of the plurality of windows may include loading at least one reactor core stack into a window of a reactor core frame. Additionally in such embodiments, loading at least one reactor core stack into a window of a reactor core frame may include loading at least one reactor core stack that includes two or more reactor core elements. Additionally in such embodiments, loading at least one reactor core stack into a window of a reactor core frame may include loading at least one reactor core stack that includes two or more reactor core elements that are connected in parallel. In such embodiments, loading at least one reactor core stack into a window of a reactor core frame may include loading at least one reactor core stack that includes two or more reactor core elements that are connected in series.

In embodiments, two or more reactor core components are secured within the fluid tight fluid reactor housing. In embodiments, the method may further include securing at least one reactor core accessory within the fluid tight fluid reactor housing. In such embodiments, the at least one reactor core accessory is a heat exchanger.

In another aspect of the present disclosure a method of compositionally transforming a primary fluid is provides and includes: flowing a primary input fluid through at least one first input port of a fluid reactor and into and through a primary fluid input manifold, where the primary fluid input manifold includes at least one primary fluid line and at least one primary fluid input cavity; flowing the primary input fluid over a reactor core component containing at least one reactor core element located in fluid communication with the primary fluid cavity such that the primary input fluid enters at least one fluid channel located within the at least one reactor core element; flowing a secondary input fluid through at least one second input port of the fluid reactor and into and through a secondary fluid input manifold, where the secondary fluid input manifold includes at least one secondary fluid line and at least a portion of at least one secondary fluid cavity; guiding the secondary input fluid from the secondary fluid cavity across the at least one reactor core element in a manner perpendicular to a direction of flow of the primary input fluid; interacting the primary input fluid with the secondary input fluid whereby the primary input fluid is compositionally changed into a primary output fluid and the secondary input fluid is compositionally changed into a secondary output fluid; flowing a primary output fluid out of the at least one fluid channel and into and through a primary fluid output manifold, where the primary fluid output manifold includes at least one primary fluid line and at least one primary fluid output cavity; flowing the primary output fluid from the primary fluid output manifold through at least one primary fluid output port from where it exists the fluid reactor; flowing a secondary output fluid out of the at least one reactor core element and into and through a secondary fluid output manifold, where the secondary fluid output manifold includes at least one secondary fluid line and at least a portion of at least one secondary fluid output cavity; and flowing the secondary output fluid from the secondary fluid output manifold through at least one secondary fluid output port from where it exits the fluid reactor. In embodiments, the primary input fluid may be blood, brackish water, salt water, water, or an industrial fluid.

### BRIEF DESCRIPTION OF THE DRAWINGS

The above and other aspects, features, and advantages of the present manufacturing methods for fluid reactors will become more apparent in light of the following detailed description when taken in conjunction with the accompanying drawings in which:

FIG. 1 schematically depicts a reactor core frame having of a bottom frame part and top frame part;

FIGS. 2A and 2B schematically depicts a partial cross-sectional view of a reactor core component;

FIG. 3 shows a simple fluid reactor incorporating a single reactor core component;

FIG. 4 shows a complex fluid reactor with a standalone fluid reactor unit housing;

FIG. 5 shows a reactor core accessory in the form of a heat exchanger plate;

FIG. 6 schematically depicts an exploded view of a reactor core accessory sandwiched between two reactor core components, where the reactor core accessory is a heat exchanger made of flat sheet materials;

FIG. 7 shows a reactor core accessory in the form of an electrically powered heater;

FIG. 8 schematically depicts a reactor core frame made of parts made from flat sheet material;

FIG. 9 schematically depicts the cross-sectional view of a reactor core component utilizing a frame 10 as shown in FIG. 8;

FIG. 10 shows a complex fluid reactor with a standalone fluid reactor unit housing for a three-port fluid reactor;

FIG. 11 is a flow chart showing the manufacturing steps for building reactor core components;

FIG. 12 is a flow chart showing the manufacturing steps for building a complex fluid reactor; and

FIG. 13 shows a fluid reactor in accordance with another embodiment of the present disclosure.

### DETAILED DESCRIPTION

Particular embodiments of methods and systems used to build fluid reactors incorporating a reactor core that has at least one reactor core component and, optionally, at least one compatible reactor core accessory are described hereinbelow with reference to the accompanying drawings; however, it is to be understood that the disclosed embodiments are merely exemplary of the disclosure and the present methods may be embodied and/or augmented in various forms. Each reactor core component includes at least one reactor core subcomponent that contains, in general, at least one free standing reactor core element having multiple fluid channels that are surrounded by an open-pore cellular network material having a bi-continuous tortuous phase structure. The embodiments described herein primarily focus on the specific case where the reactor core element is a c-VACNT reactor core element. In embodiments, such c-VACNT reactor core element are coated. Furthermore, embodiments depicted herein may incorporate a particular number of reactor core stacks that have a particular width to length ratio and height and are spatially arranged in a particular layout; however, it is to be understood that these depictions are merely exemplary of the disclosure and are not intended to be limiting. One skilled in the art will readily recognize from the following discussion that alternative embodiments of the designs and methods illustrated herein may be employed without departing from the principles of the present disclosure described herein, including, in particular, the application of the below teachings to (i) non c-VACNT reactor core elements, (ii) various alternative geometries of reactor core elements and/or reactor core stacks and their spatial layout, and/or (iii) various forms of sealing types and methods, such as taught by the '375 application. Furthermore, while specific reactor core accessories are described in greater details and may be beneficial for certain fluid reactor applications, this application is intended to include modifications thereof and other accessories that can be beneficial for other fluid reactor applications, as will be apparent to those skilled in the art based on the hereinbelow teachings.

Therefore, specific structural and/or functional details, including without limitation order, quantity, and/or types of parts and/or process steps, disclosed herein are not to be interpreted as limiting, but merely as a basis for the claims and as a representative basis for teaching one skilled in the art to variously employ the concepts of the present disclosure in virtually any appropriately detailed structure and/or arrangement. Any patents, published patent applications, and non-patent public documents or publications mentioned herein are incorporated by reference herein in their entireties.

Commonly owned U.S. Provisional Patent Application Ser. No. 62/754,375 (hereinafter, “the '375 application”) discusses reactor core elements that, in general, are built as a free-standing structure having multiple fluid channel perforations surrounded by an open-pore cellular network material having a bi-continuous tortuous phase structure. Carbon-infiltrated vertically aligned carbon nanotube (sometimes referred to herein as “c-VACNT”) reactor core elements, as also described in detail in the '375 application, are a specific embodiment of these reactor core elements that have sufficient mechanical strength to survive a drying step after liquid exposure without changing its shape and that include a plurality of parallel aligned through-perforations (hereinafter, “fluid channels”), each surrounded by a nano-porous sidewall, separating each fluid channel cross-sectional area from a porous c-VACNT material that is comprised of a nanocarbon-based, open-pore cellular network having a tortuous bi-continuous phase structure. “Tortuous,” as used herein, refers to a phase that requires numerous and frequent changes in direction when moving from one point in a phase to another so that, on average, the shortest line physically connecting two points in a phase, while traveling only inside the phase volume, is much longer than the line of sight distance between the two points. “Continuous,” as used herein, generally refers to a phase wherein all points are directly mechanically connected; therefore, for any two points within a continuous phase, there exists a path which connects the two points without leaving the phase. “Bi-continuous,” as used herein, refers to a material containing two separate phases such that each phase is continuous, and in which the two phases are interpenetrating, such that it is impossible to separate the two structures without destroying at least one of them. The c-VACNT material is comprised of a void phase and a solid phase structure made of carbon nanotube ligaments “spot-welded” together by a thin carbon film having a tunable strength and stiffness, determined in part by the thickness of the “spot-welding” carbon film. The volume of the void phase of a c-VACNT reactor core element is adjustable by process parameters. The content of the '375 application is hereby incorporated by reference herein in its entirety.

As discussed in the '375 application, a reactor core may include at least one reactor core subcomponent, where a reactor core subcomponent can be (i) a reactor core element, (ii) a reactor core stack, (iii) a parallel reactor core stack, or (iv) a serial and/or parallel combination thereof. The most basic reactor core subcomponent is a free-standing reactor core element, or more specifically a c-VACNT reactor core element. Multiple reactor core elements can be combined in series to form a reactor core stack. Furthermore, multiple reactor core elements, multiple reactor core stacks, or a combination of both reactor core elements and reactor stacks can be combined in parallel to form a parallel reactor core stack. A reactor core is enclosed in a fluid reactor unit housing.

The reactor core input surface is connected in a sealed manner to at least one input port through an input manifold having at least one input line and the reactor core output surface is connected in a sealed manner to at least one output port through an output manifold having at least one output line. In some applications, it is beneficial to make these fluid connections in such a manner that (i) the volume of at least one fluid (i.e., primary or secondary fluid) inside a respective fluid reactor unit housing is reduced, (ii) the pressure drop across the fluid reactor is sufficiently low for the fluid reactor's intended purpose for the primary and/or the secondary fluid, and/or (iii) the temperature of the primary input fluid is different from the primary output fluid.

For example, when such fluid reactors are used as oxygenators and have blood as their primary fluid, it is desirable to (i) keep the priming volume low, (ii) have a blood flow pressure drop across the device of approximately no more than a typical human heart output (i.e., <120-150 mmHg), (iii) minimize the extracorporeal foreign surface area that blood is in contact with, (iv) have a laminar blood flow path, (v) have a low accumulated shear stress to minimize platelet activation, (vi) have an antithrombic blood contact surface, (vii) have a minimum amount of anticoagulant into the blood stream, (viii) minimize the amount of blood transfusion, (ix) minimize bubble formation, and/or (x) not have an external blood pump. These oxygenator design goals help to reduce the occurrence of hemodilution, hemolysis, and/or other blood damage induced organ and/or vascular deterioration, malformation, and/or inflammation for patients connected to a blood circuit incorporating an oxygenator. In addition, it is sometimes desirable to heat or cool a patient by changing his/her blood temperature, such as during a cardiopulmonary bypass surgery, which typically uses an oxygenator in a heart-lung machine. Typical oxygenators have an operating pressure of at least 50 mmHg. Herein, however, “oxygenators” shall also refer to devices with a lower than 50 mmHg operating pressure. For brevity, “oxygenator” shall also refer to artificial lungs and portable artificial lungs in any form, including, but not limited to, portable artificial lungs running on air instead of oxygen, portable artificial lungs that use the heart as a pump rather than an external pumping device, and wearable pumpless air-consuming artificial lungs (PAALs).

In addition, to enhance the performance and/or utilization time of c-VACNT-based fluid reactors for selective applications (for example, blood oxygen saturation increase and CO₂ content reduction, various forms of membrane distillation (such as water desalination), hemodialysis, etc.), it is desirable to coat reactor core elements with at least one additional material to increase, at least locally, its hydrophobicity, thus reducing water or blood plasma leakage through the fluid channel sidewalls and/or from the reactor core element input or output surface into the void phase of the open-pore cellular network material. Other times, it is desirable to increase, at least locally, the hydrophilicity of reactor core elements, thus enabling the void phase to fill easier with water-based fluids and related suspensions. Coatings, which may be in the form of a conformal thin film, are ideally applied locally to selected locations of a reactor core element. For example, silane type materials (FOTS, FDTS, etc.) and fluorocarbon type materials (PTFE, Teflon, etc.) are traditionally used in MEMS fabrications to create localized 0.5-5 μm thick film regions with low stiction and/or friction properties, as well as localized hydrophobic surface regions. However, the combination of a hydrophobic conformal ligament surface coating with the nano-porosity of the c-VACNT material, for example, can make the resulting outer surfaces of a reactor core element superhydrophobic (contact angle (“CA”)≥) 150°, thus making it very difficult to nearly impossible for the surface to form a chemical bond with certain sealing/bonding/gluing materials (for example, some UV curable glues). However, for such cases, a sealing method is still required to allow the manufacturing of a water/blood plasma tight seal. In embodiments, it may be beneficial to create reactor core elements that are hydrophobic, superhydrophobic, hydrophilic, superhydrophilic, oleophobic, oleophilic, polar, and/or non-polar.

Contrary to the '375 application, which defines a reactor core stack as at least two reactor core elements stacked in series, to simplify the teachings below, a reactor core stack as that term is used herein means a stack of reactor core elements made of at least one reactor core element.

A fluid reactor is made of at least one fluid reactor unit having (i) a fluid reactor unit housing enclosing a reactor core, (ii) at least one primary fluid input port and fluid line, (iii) at least one primary fluid output port and fluid line, and (iv) at least one secondary fluid port and fluid line. In embodiments, each fluid reactor unit housing can optionally include at least one additional port and fluid line selected, for example, from: a venting port and fluid line, a sampling port and fluid line, a chemical injection port and fluid line, a primary fluid input temperature monitor port and fluid line, a flow rate sensing port and fluid line, a primary fluid output temperature monitor port and fluid line, an internal heat exchanger temperature monitoring port and fluid line, a venous blood dissolved oxidation monitoring port and fluid line, an arterial blood dissolved oxidation monitoring port and fluid line, a venous blood dissolved carbon dioxide monitoring port and fluid line, an arterial blood dissolved carbon dioxide monitoring port and fluid line, a selective chemical compositional monitoring port and fluid line for a primary input fluid, a selective chemical compositional monitoring port and fluid line for a primary output fluid, a bubble detection port and fluid line, a fluid conductivity measuring port and fluid line, a dissolved gas concentration measuring port and fluid line, an input pressure monitoring port and fluid line, an output pressure monitoring port and fluid line, an electrical feed through port and fluid line, a sensing port and fluid line, a bubble removal port and fluid line, a fluid insertion port and fluid line, a fluid removal port and fluid line, a fluid sample removal port and fluid line, a cooling/heating fluid input port and fluid line, a cooling/heating fluid output port and fluid line, and other ports and fluid lines useful for specific fluid reactor applications.

In embodiments, (for example, a portable artificial lung type fluid reactor) at least one external fluid reactor unit accessory is incorporated and/or attached to the external side of the fluid reactor unit housing, where such accessories can be selected from the non-limiting group of a fluid pump, a blood pump, an air blower, a gas flow regulator, a gas mixture adjuster, a signal transmitter, a signal receiver, a controller, a battery pack, a wireless battery recharger port, a light transmitter and receiver (for example, to measure one or more blood parameters, such as the Terumo CDI 500 or 550 system), a flow rate sensor, a bubble detection sensor, an electrical connector, and other accessories useful for a specific fluid reactor application. In embodiments, where one of the fluid reactor unit accessories is an electrical connector, such an external electrical connector connects to an internal reactor core accessory selected from the non-limiting group comprised of a heater, a sensor, a temperature sensor, a pH sensor, a salinity sensor, an internal pump, a steering device, etc. In embodiments, where one of the fluid reactor unit accessories is a controller, such a controller may process input data and generate output signals to maintain optimum primary output fluid processing conditions depending on (i) a change in the performance of a reactor core over time (pressure drop change, oxidation efficiency change, etc.), (ii) a primary input fluid composition change (blood oxidation, hematocrit level change, etc.), (iii) secondary fluid parameters (gas mixture concentration, flow rate change, etc.), (iv) output demand signal (higher output level needed for anticipated increased physical activity, etc.), and/or (v) other factors relevant to a specific fluid reactor application. In embodiments, a heat exchanger and/or electrically powered heater and/or cooler may be located outside the fluid reactor housing but in close proximity to such housing in order to minimize heat loss effects.

At a minimum, a reactor core has a reactor core input surface and a reactor core output surface and includes at least one reactor core assembly. At least one reactor core assembly present in the reactor core is comprised of at least one reactor core component. A reactor core component includes a reactor core frame and at least one reactor core subcomponent, where a reactor core subcomponent can be selected from (i) a reactor core element, (ii) a reactor core stack, (iii) a parallel reactor core stack, and (iv) a serial and/or parallel combination thereof. When a reactor core component contains multiple subcomponents, such subcomponents can be connected internally in (i) a parallel manner, (ii) a serial manner, and (iii) a combination thereof.

For brevity, this application shall only discuss in detail connecting subcomponents in a parallel manner; however, it is to be understood that connecting subcomponents in a serial manner or in a combination thereof are not intended to be excluded and can be implemented by those skilled in the art based on the teachings herein.

The reactor core input surface is connected in a sealed manner to a reactor core primary fluid input manifold having at least one primary fluid input line and the reactor core output surface is connected in a sealed manner to a reactor core primary fluid output manifold having at least one primary fluid output line. Hereinafter, the primary fluid input line and primary fluid output line may be referred to as a primary fluid input cavity and a primary fluid output cavity, respectively. The reactor core primary fluid input manifold connects to the primary fluid input port and the reactor core primary fluid output manifold connects to the primary fluid output port, where the input and output ports bring primary fluid into and out of a fluid reactor unit.

In embodiments, at least one input or output manifold of the reactor core is connected in a serial or parallel manner to at least one reactor core accessory and the combination is located inside a fluid reactor unit housing. Reactor core accessories may include, but are not limited to: an electrical or liquid powered plate heater and/or plate cooler; a particle filter; an arterial blood filter; a bubble remover section; a flow rate sensor; a bubble detector; a salt, chemical, and/or particle concentration sensor; a temperature sensor; a pressure sensor; and other accessories and combinations thereof useful for a specific fluid reactor application. In embodiments, the reactor core includes at least one reactor core assembly that further includes at least one reactor core component and at least one reactor core accessory that have at least one spatially isolated fluid path and/or at least one electrically isolated current conduction path connection between them. In some embodiments, a single reactor core accessory can function on its own to provide a particular functionality to a reactor core assembly (and, in turn, to the reactor core). In other embodiments, at least two reactor core accessories are used together to provide a particular functionality to a reactor core assembly (and, in turn, to the reactor core), such as the two mating parts of a single plate cooler/heater which may be manufactured in such a manner so as to lower its production cost. In some embodiments with two or more reactor core assemblies, at least one reactor core assembly is solely made of one or more reactor core accessories, where such assembly has at least one connection line to another reactor core assembly.

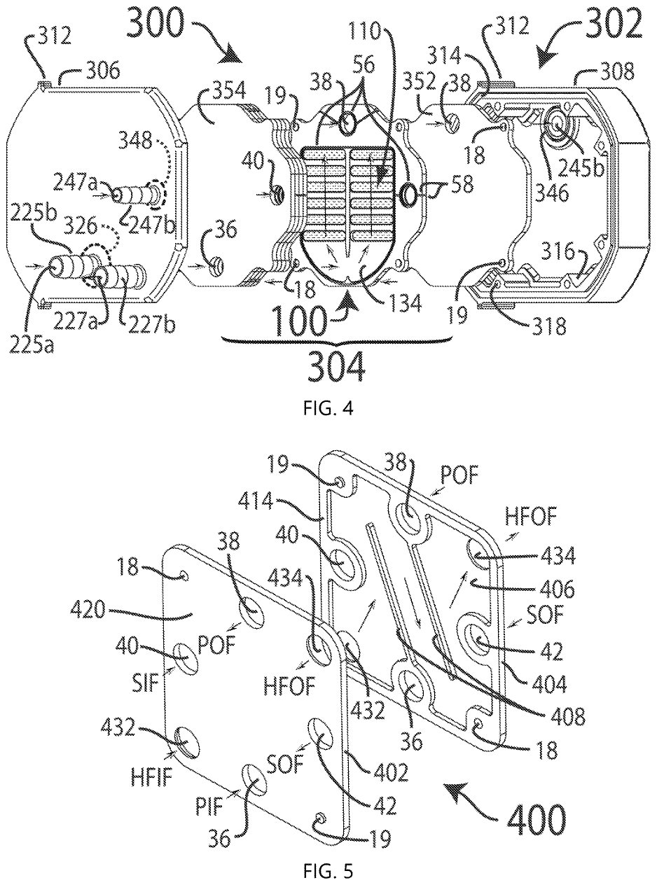

In embodiments, any reactor core assembly part can optionally contain at least one fluid sealed and spatially isolated fluid passage channel and/or at least one electrically isolated current conducting feedthrough that allows at least one fluid or electrical current to flow through the reactor core assembly part without any functional interaction. This enables the option of building of a reactor core assembly having reactor core assembly parts selected from at least one reactor core component and at least one reactor core accessory, where such assembly parts have a common input and/or output fluid and/or electrical line which connect in a serial and/or parallel manner to at least a neighboring assembly part of said reactor core assembly. These otherwise nonfunctional fluid or current passages through a reactor core assembly part can, for example, be essentially used as an assembly part bypass, thus making various reactor core assembly parts compatible with their neighboring assembly parts even if the neighbors do not have any functional interaction with one another, as will be discussed below in relation to FIG. 4.

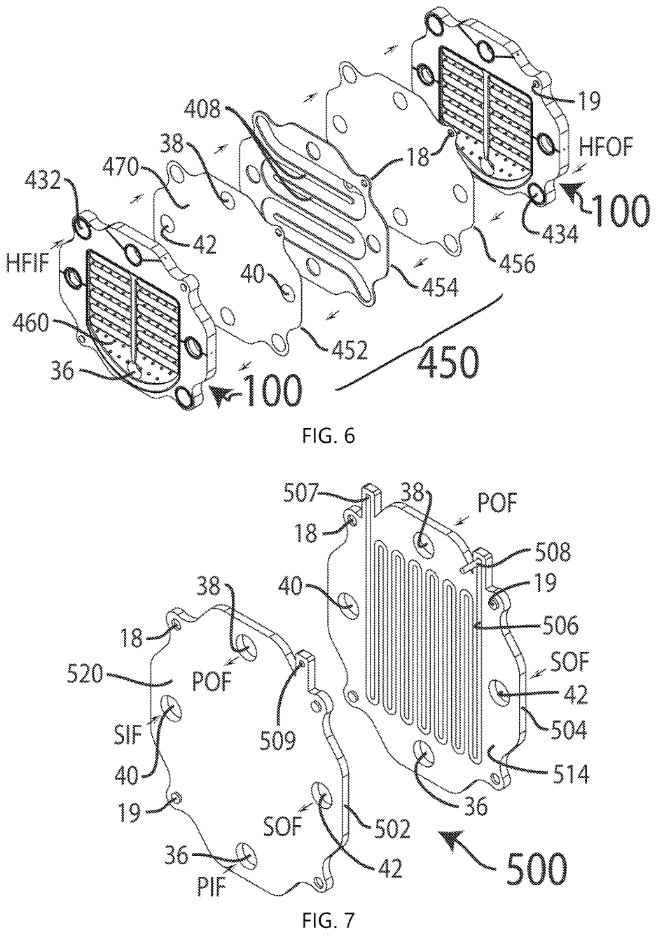

Reactor core accessories can be used, for example, for forming a portion of a fluid input or output manifold (including, not limited to, completing a primary fluid cavity), for changing the temperature of the primary fluid passing through a reactor core, for sensing at least one property of a relevant fluid, for removing trapped gas bubbles from a section of the reactor core, for passing a fluid and/or electrical current in an isolated manner from one reactor core assembly part to another, etc. In embodiments, a reactor core assembly includes a reactor core accessory in the form of a fluid heat exchanger. In some embodiments, the fluid heat exchanger (see FIG. 5 or FIG. 6) is a plate heat exchanger where the plates are exposed to a heating/cooling liquid on one side and to a primary fluid on the other side. In such embodiments, the plates are put in series with at least one reactor core component and include otherwise nonfunctional fluid passage lines that become part of the primary fluid input or output manifold for the reactor core component, thereby enabling the manufacture of a compact reactor core assembly having, for example, a small priming volume. In other embodiments (see FIG. 6), the fluid heat exchanger is made of foils and/or sheets that may contain cutout areas, as discussed below.

In some embodiments, a reactor core accessory is in the form of an electrical heating element (FIG. 7), for example in the form of a serpentine resistive heating path that is sandwiched in a fluid sealed manner between two electrically insulating plates. In some embodiments, such insulating plates are thin polycarbonate sheets with one sheet having an electrical resistive path. In one embodiment, the resistive heating path and/or an optional thermocouple is molded, printed, or plasma sprayed onto it (for example, as provided by CVD MesoScribe Technologies Corporation, Central Islip, NY, which uses a patterned thermal spray coating to write nickel alloy and other metal line patterns that can be used as electrical heaters and/or thermocouples). In other embodiments, a thin heating serpentine wire and/or a thin thermocouple are imbedded into a plastic sheet with an injection molding process with a suitably exposed connection terminal for power/sensing connections.

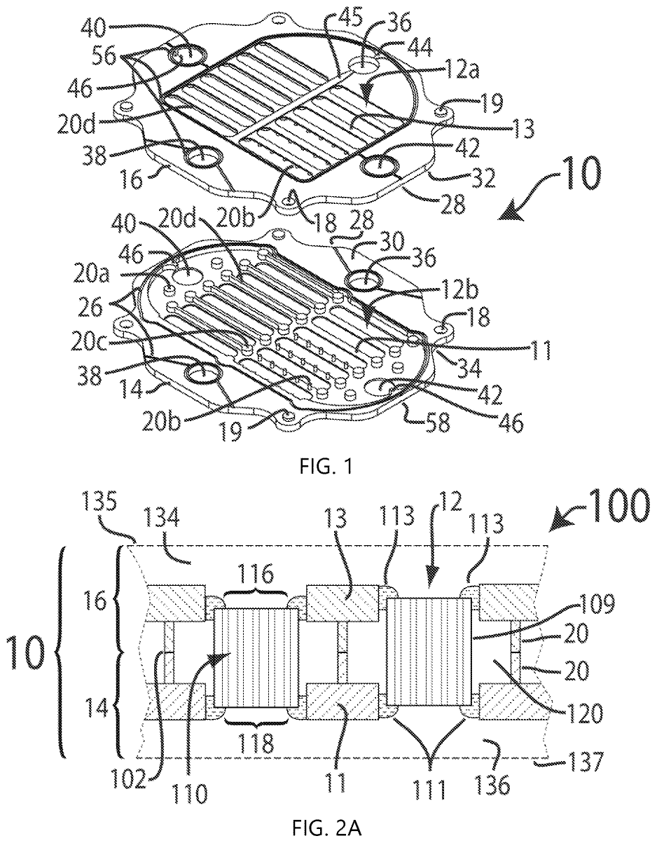

In embodiments, and as indicated in FIG. 1, a reactor core frame 10 has at least one window 12 with a bottom window frame 11 and a top window frame 13 that are separated by an air gap, where said air gap is a secondary fluid cavity. A reactor core stack is located mechanically within each window 12 and is attached in a sealed manner to the window frames 11 and 13 such that a secondary fluid can enter and/or exit each reactor core stack through an unsealed area of its outer sidewall 109 that connects its primary fluid input and output surfaces.

In embodiments, the reactor core frame 10 is a single part. In other embodiments, the reactor core frame 10 is made of at least two frame pieces that have been connected in such a manner that at least one secondary fluid cavity is formed. In some embodiments, such two frame pieces are made from a process selected from the non-limiting group of plastic injection molding, polycarbonate injection molding, stamping, casting, CNC mashing, 3D printing, or any other process capable of making the desired parts as known to those skilled in the art. 3D printing processes acceptable for making the frame pieces are those processes that are capable of producing fluid tight parts, such as an SLA printing process made with a clear or dental LT clear material sold by Formlabs or a Somos® WaterShed XC 11122 or Somos® BioClear material.

FIG. 1 shows an embodiment where a bottom frame piece 14 and a top frame piece 16 form a reactor core frame 10 after frame pieces 14 and 16 are assembled and connected together such that a fluid tight seal is formed in selected locations between the frame pieces, thereby creating at least one secondary fluid cavity. Methods of connecting the frame pieces include, but are not limited to, gluing, bonding, fusing, welding, and/or other suitable methods as known to those skilled in the art. Optional alignment features, shown in FIG. 1 in the simplified form of a hole 18 and a matching protrusion 19, can be used to facilitate the correct alignment between the bottom and top frame pieces 14 and 16 before they are bonded together to form the reactor core frame 10. The material for the frame pieces 14 and 16 should be chosen to be mechanically, thermally, and chemically stable when exposed to the various processing steps needed to build a fluid reactor unit for a given target application, as well when exposed to all the fluid during its use as an active fluid reactor, at least for the intended life of the fluid reactor. Each frame piece 14 and 16 has matching windows 12a, 12b, respectively, where the dimensions of at least one of the windows 12a, 12b are slightly larger than the reactor core stack 110 that will ultimately be located within the window 12. In embodiments, the dimensions of the oversized window 12 are between about 10 μm and about 1000 μm larger than those of a reactor core stack 110 which will ultimately populate each window 12. In embodiments, the windows 12a in frame piece 14 are a different size from the windows 12b in frame piece 16. In such embodiments, the windows 12a, 12b on at least one frame piece 14 or 16 have dimensions larger than the reactor core stack 110 that will ultimately be located within the window 12. In other embodiments, the windows 12a, 12b on both frame piece 14 and 16 have dimensions smaller than the reactor core stack 110 that will ultimately be located in the window 12. Optional standoff features can be used for flow balancing optimization, to create fluid mixing turbulence, and/or as mechanical support features that strengthen the thinner parts 11 and 13 of the frame pieces 14 and 16. FIG. 1 shows multiple versions of possible standoff features/fluid mixing turbulence generators 20 in the form of thick cylindrical pillars 20a, thin cylindrical pillars 20b, and thick cylindrical pillars 20c connected to vertical dividers 20d. Other shapes for standoffs/fluid mixers known to those skilled in the art can be chosen.

FIG. 1 also shows optional internal sealing grooves 26 which can be injected with a curable glue after the two frame pieces 14 and 16 have been mechanically aligned and assembled together. In embodiments, the frame pieces 14 and 16 are injection molded from a polycarbonate material or other equivalent functional material. In embodiments, the frame material is both sufficient UV transmissive and medical grade, for example a class VI grade biocompatible material that has been optimized for long term blood contact. First, a low viscosity UV curable glue (for example, Dymax 1120-M-UR PDS) is injected under pressure into internal sealing groove 26 from a hole 28, located at one point of the internal sealing groove 26, until the glue exits a hole 28 located at another point of the internal sealing groove 26; then, UV light is irradiated through one of the UV transmissive frame pieces 14 or 16 to cure the glue. Alternatively, a suitable thin layer of glue can be deposited or printed on selective areas of the top side 30 of the bottom frame piece 14 and/or the bottom side 32 of the top frame piece 16 prior to the mechanical assembly of both frame pieces and then allowed or made to cure. In other embodiments, the two frame pieces 14 and 16 are ultrasonically or liquid welded (for example, with a methyl chloride-based low viscosity solvent or a combination of solvent and suitable plastic filler). Any other suitable bonding method as known to those skilled in the art and as compatible with the intended fluid reactor application can be used equivalently.

FIG. 1 also shows external alignment features 34 that can be used to align a reactor core component to another reactor core assembly part and/or to align a reactor core to a fluid reactor unit housing, as will be discussed in more detail below. Furthermore, optional external alignment features, such as hole 18 and matching protrusion 19, can additionally be used to align a reactor core component to another reactor core assembly part.

In FIG. 1, each frame piece 14 and 16 has four through-holes. Through-hole 36 is part of the primary fluid input manifold and through-hole 38 is part of the primary fluid output manifold. Through-hole 40 is part of the secondary fluid input manifold and through-hole 42 is part of the secondary fluid output manifold. In embodiments, only some of through-holes 36, 38, 40, and 42 may exist. In other embodiments, frame piece 14 may have different through-holes than are illustrated on frame piece 16. In embodiments, the existing through-holes on a frame piece 14 may be arranged in a different pattern than the illustrative pattern of through-holes shown on frame piece 16. In embodiments, only one of through-hole 40 and through-hole 42 may exist. In embodiments (see FIG. 3), where a reactor core assembly is made of a single reactor core component, the frame 10 can have no through-holes 36 and 38, thus minimizing dead ended stagnant fluid flow regions, which, for example, for blood flow can lead to coagulation. In embodiments with a single reactor core component, each of through-holes 40 and 42 can be present in only one of the frame pieces 14 and 16, thus building a compact secondary fluid manifold for a 4-port fluid reactor. In embodiments, at least one of frame piece 14 and 16 may have multiple of at least one of through-holes 36, 38, 40, and 42 in order to more evenly distribute a particular fluid throughout a reactor core component or to more evenly remove a particular fluid from a reactor core component. FIG. 1 depicts though-holes 36, 38, 40, and 42 as circular in shape; however, the through-holes 36, 38, 40, and 42 may be in any shape, such as rectangular, elliptical, race-track-shaped, oval-shaped, or any other shape as known to those skilled in the art. In embodiments, the through-holes present on a frame 10 do not have to all be the same shape. In embodiments, the shape of a through-hole 36, 38, 40, or 42 is determined based on fluid dynamics and other needs for a particular fluid reactor embodiment. Also not shown in FIG. 1 are optional through-holes 264 that can be used to rivet, screw, or otherwise mechanically hold together the various reactor core assembly parts (see FIG. 3) and/or that can act as fluid or current bypass lines to transmit fluids and/or current to an assembly part without any functional interaction with any other fluid. In embodiments, frame pieces 14 and 16 are geometrically optimized for minimal shear stress accumulation of the primary fluid path, which is typically accomplished through the use of rounded corners and no dead ended flow regions.

In embodiments, the exterior portion of window frame piece 13 is the same thickness as the rest of the top frame piece 16. Similarly, in embodiments, the exterior portion of window frame piece 11 is the same thickness as the rest of the bottom frame piece 14. In such embodiments, the windows 12a on a top frame piece 16 are only open on the bottom side 32 of the top frame piece 16. Similarly, the windows 12b on a bottom frame piece 14 are only open on the top side 30 of the bottom frame piece 14. Therefore, the windows 12 are not visible from the top side of frame piece 16 nor from the bottom side of frame piece 14. In some such embodiments, the frame pieces 14 and 16 include piping that connects one window 12 to another window 12, such that all of the windows 12 within the frame 10 are serially connected. In embodiments, such piping may travel through standoff features/fluid mixing turbulence generators 20. When a reactor core component including such a frame is incorporated into a fluid reactor, primary fluid can flow serially through the reactor core component to each reactor core stack 110 before passing onto the next reactor core component in the fluid reactor or exiting the fluid reactor.

In embodiments, portions of window frame piece 13 have different thicknesses and/heights from other portions of window frame piece 13. In embodiments, portions of window frame piece 11 have different thicknesses and/heights from other portions of window frame piece 11.

The references to “top” and “bottom” made throughout the disclosure are not intended to be absolute physical references and should be understood as a temporary assignment to a reactor core frame 10 when oriented in a particular way at a particular time in the manufacturing process. Furthermore, directional references when describing a process (e.g., gluing on the top side) are not intended to be limiting and such processes can be done in other directions and orientations, such as from the bottom up, from the top down, tilted, etc. For brevity, only one such orientation is described, but any other orientations are intended to be included as well. Furthermore, whether a through-hole is part of an input fluid manifold or an output fluid manifold can be, for some designs, changed depending on the application without a significant change in performance of the fluid reactor.

The presence of flow redirection features 44 and/or flow splitters 45 within a primary fluid cavity 134 or 136 can minimize stagnant flow regions within such cavity. The presence of flow redirection features 44 and/or flow splitters 45 within a primary fluid cavity 134 or 136 can minimize priming volume. Whether or not it is beneficial to include such flow redirection features 44 and flow splitters 45 in any of the cavities 134 or 136 of a fluid reactor can depend on the fluid reactor application. In embodiments, as depicted in FIG. 1, a flow redirection feature 44 and a flow splitter 45 is available in primary fluid input cavity 134, but is not available in primary fluid output cavity 136 (not visible in the view shown in FIG. 1). Such an asymmetric case can operate to simultaneously minimize stagnant/dead-end flow zones and priming volume. This is particularly useful when blood is the primary fluid. In other embodiments, a flow redirection feature 44 and a flow splitter 45 is available in primary fluid output cavity 136, but not in primary fluid input cavity 134. In further embodiments, a similar or different flow redirection feature 44 and a flower splitter 45 is available in both primary fluid input cavity 134 and primary fluid output cavity 136. In even further embodiments, there is no flow redirection feature 44 or flow splitter 45. In other embodiments a flow redirection feature 44 is available in a cavity, while a flow splitter 45 is not available. In further embodiments, a flow splitter 45 is available in a cavity, while a flow redirection feature 44 is not available. In any embodiment where the flow redirection features 44 and/or flow splitters 45 present in a primary fluid input cavity 134 differ from those present in a primary fluid output cavity 136, reversing the flow of the primary fluid may negatively affect the stagnant flow zones.

Optionally, a flow redirection features 46 is available in a secondary fluid input manifold and/or a similar or different and/or flow redirection feature 46 is available in a secondary fluid output manifold. Where such flow redirection features 46 are available, they can be used to more equally split the flow quantity of a secondary fluid between the different reactor core stacks. In embodiments, flow redirection features 46 are used with any variety of the standoff features/fluid mixing turbulence generators 20.

In embodiments, a reactor core frame 10 is made as a single piece rather than as two separate frame pieces 14 and 16. Such frames 10 can be manufactured by any suitable manufacturing process, such as some of those processes discussed above in reference to the manufacture of multiple frame pieces, e.g., 3D printing, etc. Such a frame 10 still has at least one internal secondary fluid cavity.

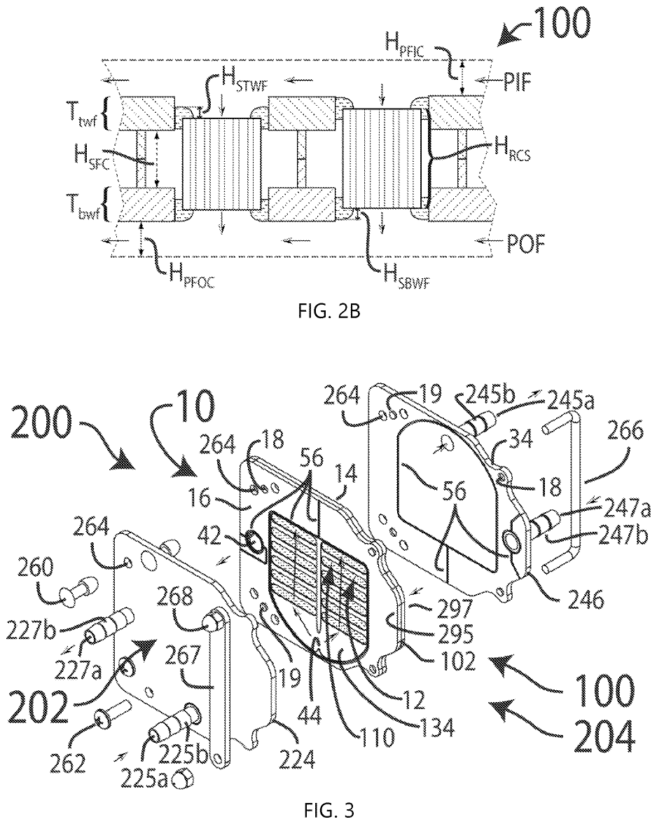

FIG. 2A and FIG. 2B show a partial cross-sectional view of a multiwindow reactor core component 100 oriented perpendicular to the longest direction of its windows 12 and parallel to a line connecting the bottom frame 14 to the top frame 16. FIG. 2A and FIG. 2B are identical except that FIG. 2A indicates the various parts of a reactor core component 100, while FIG. 2B indicates the various measurements and fluid flow directions referenced in relation to a reactor core component 100. FIG. 2A and FIG. 2B are collectively referred to hereinafter as FIG. 2. In FIG. 2, the reactor core component 100 includes a reactor core frame 10, depicted as a bottom frame piece 14 and top frame piece 16 assembled together with a bond line 102 between the mating surfaces of the frame pieces 14 and 16. The mating surfaces are typically the top surface of any available standoff features/fluid mixer 20 (as shown in FIG. 2), as well as the top side 30 of the bottom frame piece 14 and the bottom side 32 of the top frame piece 16. Reactor core stacks 110 having a height H_{RCS} are inserted into each available window 12 of the reactor core frame 10. Each reactor core stack 110 is connected to the frame 10 with a bottom corner seal 111 connecting the edge of the reactor core stack output surface 118 with a nearby bottom window frame 11, where the bottom corner seal has a height H_{SBWF} above the reactor core stack output surface 118 and the bottom window frame 11 has a thickness T_{bwf}. Each reactor core stack 110 is further connected in a sealed manner to the frame 10 with a top corner seal 113 connecting the edge of the reactor core stack input surface 116 with a top window frame 13, where the top corner seal has a height H_{STWF} above the reactor core stack input surface 116 and the top window frame 13 has a thickness T_{twf}. The thicknesses T_{bwf} and T_{twf} are chosen so that a secondary fluid cavity 120 having a height H_{SFC} is formed through which a secondary fluid can enter or exit each reactor core stack 110. In embodiments, inside a secondary fluid cavity 120, secondary fluid flows in a parallel manner, a serial manner, or in a combination thereof within a reactor core component 100. In embodiments, flow redirection features 46 and/or standoff features/fluid mixing turbulence generators 20 are used to direct secondary fluid flow through the secondary fluid cavity 120. In some such embodiments, the secondary fluid is directed to flow in a path-like manner (e.g., in a serpentine path) such that it passes by each reactor core stack 110 within the secondary fluid cavity 120 one-by-one. The secondary fluid flow path passes by at least a portion of the sidewall 109 of each reactor core stack 110. In some embodiments, the secondary fluid flow path passes by a reactor core stack 110 more than once and may come in contact with different portions of the sidewall 109 of the reactor core stack 110. While passing by at least part of the sidewall 109 of each reactor core stack 110, the secondary input fluid can flow into such reactor core stacks 110. Secondary input fluid flow into the reactor core stacks 110 may be caused by, for example, a pressure gradient or a concentration difference. Similarly, secondary output fluid can flow out of each reactor core stack 110. Secondary output fluid flow out of the reactor stacks 110 may be caused by, for example, a pressure gradient or a concentration change. The corner seals 111 and 113 can be semi-flexible or rigid, but should be of sufficient strength to prevent substantial relative movement of the reactor core stacks 110 compared to the frame 10 when exposed to fluid flows up to a given design limit.

FIG. 2 shows an embodiment where, for each reactor core stack 110 and window 12, T_{bwf}+H_{SFC}+T_{twf}≥H_{SBWF}+H_{RCS}+H_{STWF} and with H_{RCS}≥H_{SFC}. This configuration, though not necessary for all fluid reactor applications, makes it easier to ensure the seals 111 and 113 do not locally choke the primary input fluid (PIF) flow into the primary fluid input cavity 134, having a local height H_{PFIC}, or the primary output fluid flow (POF) flow into the primary fluid output cavity 136, having a local height H_{PFOC}. The primary fluid input cavity 134 is the gap formed locally between the exterior part of the top window frame 13 and a cover 135, where the cover 135 is (i) the bottom window frame 11 of a different reactor core component 100, (ii) a reactor core accessory, or (iii) the endplate, lid, or cover of a fluid reactor unit housing. Similarly, the primary fluid output cavity 136 is the gap formed locally between the exterior part of the bottom window frame 11 and a cover 137, where the cover 137 is (i) the top window frame 13 of a different reactor core component 100, (ii) a reactor core accessory, or (iii) the endplate, lid, or cover of a fluid reactor unit housing. In embodiments, cover 135 and/or 137 can be a reactor core accessory, where at least one of the functions of the reactor core accessory is to seal the respective primary fluid cavity; in embodiments, this can be the only function of such a reactor core accessory. In embodiments, the cover 135 and/or 137 can help create a serial connection between reactor core stacks 110 in a reactor core component 100. In some such embodiments, the structure of the window frame pieces 11 and/or 13 (e.g., varying heights and/or thickness of different portions of a window frame piece) may contribute to the formation of a serial connection between the reactor core stacks 110 in a reactor core component 100. To properly connect and seal various reactor core assembly parts, and to prevent or minimize local choking of the primary fluid flow near the windows 12, the seals 111 and 113 should only minimally, if at all, encroach the primary fluid cavities 136 and 134, respectively.

In embodiments, the bottom seal 111 is applied after the top seal 113 is formed by turning over the reactor core frame 10 such that the bottom window frame 11 is now oriented on top; the bottom seal 111 is then applied in the same manner as the top seal 113 was applied. Alternatively, the top and bottom seals 113 and 111 can be applied without turning over the frame 10. In some embodiments, reactor core stacks 110 are sealed to one of the frame pieces 14 or 16 prior to the frame pieces being connected together to form a reactor core frame 10.

In embodiments, each reactor core stack 110 is sealed first from the top and then from the bottom for each available window 12 sequentially, thus requiring less motion range for a given sealing equipment. Furthermore, this method allows the use of a single height adjusting control mechanism to locate each reactor core stack 110 approximately centrally into each window 12, regardless of the height of the reactor core stack 110. However, as discussed above, the maximum height of a reactor core stack for a given reactor core frame 10 is ideally chosen by the constraints T_{bwf}+H_{SFC}+T_{twf}≥H_{SBWF}+H_{RCS}+H_{STWF} and H_{RCS}≥H_{SFC}, thus preventing or minimizing any local flow choking of the primary fluid input cavity 134 and the primary fluid output cavity 136. Such an embodiment is, for example, useful for a manual sealing station or for a small production volume operation.

In another embodiment, all reactor core stacks 110 are placed on a prepositioned shelf and then sealed, or at least tack welded, from the top at their top edge (i.e., their primary fluid input surface 116). Then, the frame 10 is turned over and the all reactor core stacks 110 are sealed, or at least tack welded, from the top at their bottom edge (i.e., their primary fluid output surface 118). If the reactor core stacks 110 were initially only tack welded on each side, then they can be fully sealed subsequently. While such a method typically allows for a given frame 10 to have a smaller height tolerance range for the reactor core stack height H_{RCS}, it can speed up the production rate of reactor core components through automation, while keeping the sealing hardware system simpler.

FIG. 2 depicts two sealed reactor core stacks 110, where one stack 110 is shorter and is not vertically centered in the window 12 and the other stack 110 is taller and is more vertically centrally located in the window 12. Generally, as long as each reactor core stack 110 is properly sealed at its top and bottom corners and the heights H_{SBWF} and H_{STWF} of seals 111 and 113, respectively, do not significantly exceed the external surface of window frames 11 and 13, respectively, the vertical location of a reactor core stack 110 minimally impacts, if at all, the performance of the fluid reactor incorporating the reactor core component 100 with such stacks 110. However, the thicker the window frames 11 and 13 (i.e., the thicker T_{bwf} and T_{twf}), the larger the primary fluid volume (i.e., priming volume) of the particular reactor core component 100. Thicker window frames 11 and 13 tend to be easier to manufacture than thinner window frames 11 and 13. Therefore, for applications where the size of the priming volume matters, for example, blood oxygenation for a heart-lung machine, a compromise needs to be made between the allowed minimum and maximum height H_{RCS} and the window frame thicknesses T_{bwf} and T_{twf}.

In still a further embodiment, first the height H_{RCS} of each reactor core stack 110 is measured. Based off of this learned height H_{RCS} information, each reactor core stack 110 is placed on a height adjustable shelf such that each stack 110 is approximately vertically centered in its respective window 12 while still abiding by the parameters that T_{bwf}+H_{SFC}+T_{twf}≥H_{SBWF}+H_{RCS}+H_{STWF} and H_{RCS}≥H_{SFC} for each stack 110. In further embodiments, the learned height values HRS are used to more optimally place a sealing system's sealing generation mechanism, e.g., a nozzle. The height H_{RCS} for each reactor core stack 110 can be measured, for example, with a calibrated video camera connected to a suitable vision acquisition system, with a laser micrometer, with a single point or line laser scanner, with a digital micrometer that has movable sensing probes, with an air actuated traveling contact probe, or with any other method as known to those skilled in the art. Various methods can be used to move reactor core stacks 110 from their holding cartridge and/or height measuring station and place them into the next processing location, such as by doing so manually or with a robotic arm equipped with a spring-loaded mechanical or vacuum powered pickup hand. By first measuring the height H_{RCS} of a reactor core stack 110, those stacks 110 with a height H_{RCS} outside the targeted minimum-maximum height range of a specific reactor core frame 10 can be rejected and/or put aside for processing in a different frame 10 where such sized stacks 110 are appropriate or reworking to a suitable height as discussed in the '375 application. By first measuring reactor core stacks 110 and then positioning them in the frame 10, reactor core components 100 can be built within production specifications for its given height range H_{RCS}, thus possibly lowering overall production cost.

As depicted in FIG. 2, the top and bottom seals 113 and 111 isolate the secondary fluid cavities from the primary fluid cavities. In one embodiment, a robotized (3, 4 or 5 axis) pressurized needle dispensing system or a contactless glue dispensing system (such as the PICOR Pulse™ by Nordson EFD) deposits UV curable glue by first following the path between the top outer edge of the reactor core stack 110 and the inner edge of the top window frame 13 and then doing the same to the bottom window frame 11, optionally after the frame 10 has been turned over. In embodiments, a video camera and vision acquisition software and/or a laser point scanner or 2D laser scanner (for example, Keyence's LJ-X series) guide at least one curable glue or polymer dispensing tip or jetting valve along an optimal sealing path, where the curable glue or polymer is optionally heated by the dispensing tip or jetting valve. In other embodiments, the sealing material is applied manually with a pressurized syringe. In embodiments, the deposited sealing material is a high viscosity UV curable glue (for example, Dymax 1405-T-UR-SC PDS). In other embodiments, the seals are made from the same material as the frame 10 (e.g., polycarbonate). In embodiments, the seals are deposited with a robotized hot dispensing tip connected to a heated liquid polymer reservoir, such as those found inside a 3D printing system capable of printing small lines of polycarbonate. In embodiments, the seal dispensing system is used in combination with a visually aided and/or laser scanning guiding system in order to improve the positional accuracy and minimize the width of the sealing line so that close to the maximum fluid channel zone area of the reactor core stacks 110 can be utilized for fluid processing.

In embodiments, a seal is applied via a melting process rather than a gluing process, or via another suitable additive material deposition process. Applying a seal with a melting process can reduce or eliminate the introduction of another material into the sealing mixture, thereby potentially simplifying medical related testing requirements, reducing material interaction complications or aging effects, and/or reducing the effects of long-term exposure to fluids (e.g., minimizing swelling of seal or material property losses). In embodiments, frames 11 and 13 are designed and made in such a manner that additional material is available near the edge of windows 12, e.g., a lip, that can be used to fill the gap between the reactor core stack 110 and the frames 11 and 13 with a suitable thermal short-term local melting process. In embodiments, the melting is done by ultrasound, infrared heat, a directed light beam, an electromagnetic energy beam, a suitably shaped heated probe, or other means known to those skilled in the art. For example, melting may be done by a partially masked laser, flash lamp, or IR heater powered light beams, which types of electromagnetic energy beams. In embodiments, a reactor core stack 110 is corner sealed into a window 12 via a local melting process by slightly pushing a suitably shaped hollow hot tip into the frames 11 and 13, thus temporarily and quickly locally melting the frame material so it can connect with the side edge of the reactor core stack 110 and form a seal.

In embodiments, and as shown in FIG. 1, to make it easier to seal reactor core stacks 110 into the windows 12, the windows 12 and the matching reactor core stacks 110 have a corner radius. This is particularly useful in embodiments where the reactor core stacks 110 have smooth edges (i.e., no notches or protrusions, as discussed in the '375 application). In embodiments, the corner radius is approximately ½ to ¼ of the width of the reactor core stack 110. For example, to maximize yield for a 4″ Si wafer used to manufacture reactor core elements, as discussed in the '375 application, and to locate a single reactor core element having a length L≈30 mm and a width W≈2.5 or 5 mm, a corner radius R≈1.25 or 2.5 mm can be chosen. In embodiments, the reactor core stacks 110 and windows 12 have nearly square corners. In further embodiments, the reactor core stacks 110 and windows 12 have square corners.

The optional standoff features/fluid mixing turbulence generators 20 located between two adjacent windows 12 together with the rounded corners of the reactor core stacks 110 can be engineered and spaced in such a manner as to enhance the secondary fluid extraction and/or delivery for frames 10 having multiple rows and columns of windows 12.

In embodiments, the gap between a reactor core stack 110 and the edge of a window 12 is about 25-500 μm and the sealing path width is about 100-600 μm, thus balancing mass manufacturability with automated sealing systems and performance (e.g., priming volume and flow capacity) for a fluid reactor unit incorporating such a reactor core component 100.

In embodiments, a hydrophobic coating is applied to a reactor core element before it is made into a reactor core stack 110 and/or glued into a window 12 of a frame 10. For many material combinations, this can prevent the formation of a strong chemical bond between a chosen sealant material and a hydrophobically coated reactor core stack 110. In embodiments, if a seal is applied via a hot melting sealing method, when the seal cools and solidifies, a small mechanical gap can form. Similarly, in embodiments where a seal is applied with a reactable or curable material, when the sealing material reacts or cures, a small mechanical gap can form. If the contact angle CA of the hydrophobically coated reactor core stack 110 is high enough (i.e., greater than about) 90°, then the capillary repulsion can be sufficiently high to prevent liquid from penetrating small gaps at normal operating pressures suitable for a specific fluid reactor application. In embodiments, such small gaps can be less than about 10 μm; in other embodiments, less than about 2 μm, and in further embodiments, less than about 1 μm. For example, a 10 μm or 200 nm gap on a material that has a contact angle of CA≈150° creates a capillary repulsion force on the order of 3.6 PSI or 180 PSI (i.e., much higher than the operational pressure of most fluid reactor applications), thereby effectively enabling the formation of a liquid tight seal, even if the sealing material and the reactor core stack 110 do not chemically bond well, and even if the sealing material mechanically changes (e.g., locally swells/shrinks slightly, i.e., less than about 10 μm) over the fluid reactor's manufacturing process and/or usage life.

In embodiments, a PTFE-like coating (as discussed below) on the c-VACNT type reactor core can result in an initial contact angle of CA≥150° and can be manufactured in such a way that the contact angle CA stays above 90° for multiple weeks of exposure to clean water (see TechConnect 2019 poster titled “c-VACNT™ enabled Fluid Reactor Innovations: a NanotoMacro™ transformation”: DOI: 10.13140/RG.2.2.30775.06567, hereinafter referred to as “the TechConnect poster”). Even when used in conjunction with Dymax 1405-T-UR-SC PDS UV curable glue, which is not certified for long term water exposure and has a 1.4% water absorption in 24 hours, an effective fluid tight seal can be obtained that may last at least over a few weeks of operation, as shown in the TechConnect poster, due to the mechanical and capillary repulsion forces present. In further embodiments, the hydrophobic nature of such treated c-VACNT reactor core elements can survive at least 11 weeks of water exposure. In embodiments, the hydrophobic nature of such treated c-VACNT reactor core elements can be preserved after at least one week of water exposure. In further embodiments, the hydrophobic nature of such treated c-VACNT reactor core elements can be preserved after at least one month of water exposure. In even further embodiments, the hydrophobic nature of such treated c-VACNT reactor core elements can be preserved after at least one year of water exposure.

When present, a through-hole 36 in reactor core component 100 allows some of the primary fluid arriving from an upstream reactor core assembly part or fluid reactor housing piece to bypass the primary fluid input cavity 134 of reactor core component 100. When present, a through-hole 38 in reactor core component 100 allows primary output fluid arriving from an upstream reactor core assembly part to combine with the primary output fluid exiting the primary fluid output cavity 136 of reactor core component 100.

As depicted partially in FIG. 1, the flow splitter 45 and/or redirection features 44 and 46, when available, can help to minimize stagnant flow zones and/or aid flow distributions and laminar flow pattern development. For example, it may be beneficial to add such flow splitter 45 and/or flow redirection features 44 and/or 46 to reactor core components used to build oxygenators incorporating multiple rows and/or columns of reactor core stacks 110, since (i) minimizing dead end flow paths in oxygenators reduces the occurrence of blood coagulation and (ii) mixing secondary fluid flow lines enhances delivery of oxygen and/or removal of carbon dioxide from blood. FIG. 1 also shows optional external sealing grooves 56 on the external surface of the frame 10 that can be filled with an injectable glue, via hole 58, to allow localized sealing and bonding to another flat surface, such as for sealing and bonding one reactor core component 100 to another reactor core assembly part, thereby completing either a primary fluid input cavity 134 or a primary fluid output cavity 136. Similar to a hole 28, any excess glue injected into a sealing grooves 56 will exit the groove via a different hole 58 located at another point of the sealing groove 56.

FIG. 3 depicts an embodiment of a simple fluid reactor unit 200 made of a fluid reactor unit housing 202 containing a reactor core 204, where the reactor core 204 is a simple single reactor core component 100 including a reactor core frame 10 formed from a bottom frame piece 14 and a top frame piece 16, where the frame 10 has twelve windows 12 each containing a reactor core stack 110. In other embodiments, the frame 10 may have a different number of windows 12 and/or the windows 12 may be arranged differently. The housing 202 is made of sealed combinations of (i) an endplate 224 with primary fluid input port 225a and line 225b and secondary fluid output port 227a and line 227b, (ii) an endplate 246 with primary fluid output port 245a and line 225b and secondary fluid input port 247a and line 247b, and (iii) the outer edge of the reactor core component 100. Seals between the endplate 224 and the top surface 295 of the component 100 and seals between endplate 246 and the bottom surface 297 of the component 100 hold the fluid reactor housing 202 together. Furthermore, once the seals are in place, a primary fluid input cavity 134 is formed between endplate 224 and the top window frame 13 and a primary fluid output cavity 136 is formed between the endplate 226 and the bottom window frame 11. Optional external sealing grooves 56 can be used, as discussed above, to create a seal in selected areas between two surfaces. In embodiments, the exterior surface of a top window frame 13 is level with the exterior surface of its top frame piece 16. In embodiments, the exterior surface of a bottom window frame 11 is level with the exterior surface of its bottom frame piece 14. In embodiments where a window frame is level with its frame piece, the neighboring cover 135 or 137 may include a recessed area so that a primary fluid cavity 134 or 136 can still be formed. In embodiments, the cover 135 or 137 with a recessed area is endplate 224 or 226. In embodiments, the cover 135 or 137 may have a recessed area, regardless of whether or not the window frame and frame piece of its neighboring reactor core component 100 are level.

In embodiments, a primary fluid cavity 134 or 136 has a non-constant height cross-sectional profile. In embodiments, the height of the cross-sectional profile changes along the length of the cavity 134 or 136 with a taller height H_{PFIC} or H_{PFOC} near the respective fluid entrance or exit area and a shorter height further from such area. This can help, for example, to balance the flows into or out of reactor core stacks 110, particularly when a reactor core component 100 contains an arrangement of multiple reactor core stacks 110.

In the embodiment shown in FIG. 3, the endplate 224 has a primary fluid input port 225a and a secondary fluid output port 227a, each of which is the threshold into a fluid line 225b and 227b, respectively. The endplate 246 has a primary fluid output port 245a and a secondary fluid input port 247a, each of which is the threshold into a fluid line 245b and 247b, respectively. In embodiments, the ports and fluid lines may be arranged differently, including being located on different endplates and/or being located in different locations on a specific endplate. In FIG. 3, all fluid lines are depicted as barbed hose connectors extending from their respective endplate. However, the fluid lines could be made in any other form capable of transporting fluid from outside the housing 302 into the housing 302 suitable for a fluid reactor application. For the embodiment shown in FIG. 3, the combination of fluid line 225b and the primary fluid input cavity 134 is the primary fluid input manifold and the combination of fluid line 245b and the primary fluid output cavity 136 is the primary fluid output manifold. Furthermore, in FIG. 3, the combination of fluid line 247b, through-hole 40, and a portion of the secondary fluid cavity 120 is the secondary fluid input manifold. The combination of fluid line 227b, through-hole 42, and a portion of the secondary fluid cavity 120 is the secondary fluid output manifold. For this embodiment, the secondary fluid cavity 120 is part of both the secondary fluid input manifold or the secondary fluid output manifold. In the embodiment depicted in FIG. 3, through-hole 42 is only located on frame piece 16 and through-hole 40 is only located on frame piece 14.

In embodiments, the combination of fluid line 225b, all through-holes 36, and all primary fluid input cavities 134 is the primary fluid input manifold. In embodiments, the combination of fluid line 245b, all through-holes 38, and all primary fluid output cavities 136 is the primary fluid output manifold. In embodiments, the secondary fluid input manifold includes fluid port 247b and all through-holes 40. In embodiments, the secondary fluid output manifold includes fluid port 227b and all through-holes 42. In embodiments, each secondary fluid cavity 120 can also be part of the secondary fluid input manifold and/or the secondary fluid output manifold, depending on the fluid reactor application. In embodiments for 3-port fluid reactors, each secondary fluid cavity is part of only one type of secondary fluid manifold. In embodiments, any air gap region between a reactor core and a housing can be part of at least one of the manifolds. In embodiments, endplate 224 and/or 226 can have internal pathways through which primary or secondary fluid travels, pools, and/or splits. Such pathways are part of the appropriate fluid manifold.

Optionally, additional mechanical structural reinforcements may be added to the fluid reactor unit housing 202 and/or reactor core assembly parts, for example, to minimize the chance of a mechanical failure of the seals. A mechanical failure could occur due to, for example, a mechanical shock to the fluid reactor unit 200; a pull on a fluid line 225b, 245b, 227b, or 247b; or an over-pressure condition. Such optional additional mechanical structural reinforcements could be, for example, (i) rivets 260, (ii) screws 262, (iii) through-holes 264, (iv) a U-bolt 266 with matching bracket 267 and nuts 268 interlocking with external alignment features 34, or (v) any other reinforcement as known to those skilled in the art.

In embodiments, the endplates 224 and 246, the reactor core frame 10, and all related seals are made of biocompatible polycarbonate, which may be suitable, for example, for a blood oxygenation application. In other embodiments, the endplates 224 and 246, the reactor core frame 10, and all related seals are made of a material having a glass transition temperature significantly above 100° C., which may be suitable, for example, for a membrane distillation application. In embodiments, the usable temperature of the material from which the reactor core frame 10, endplates 224 and 246, and any related seals are made is at least 150-250° C. In embodiments, the endplates 224 and 246 and the reactor core frame 10 are made via an injection molding process. In embodiments, the fluid lines 225b, 227b, 245b, and/or 247b can be molded directly onto their respective endplates 224 or 246. In other embodiments, the fluid lines 225b, 227b, 245b, and/or 247b are molded as separate parts and then glued onto their respective endplates 224 or 246 by any means known to those skilled in the art, such as ultrasonic welding or UV curable glue. In embodiments, the housing seals are done sequentially, e.g., by first sealing the reactor core component 100 with endplate 224 and subsequently sealing the reactor core component 100 with the endplate 226 or vice versa, thereby completing the primary fluid input manifold, the primary fluid output manifold, the secondary fluid input manifold, and the secondary fluid output manifold. Such seals can be formed, for example, with UV curable glue or by ultrasonic welding.

In the embodiment show in FIG. 3, the primary input fluid flows through the input port 225a into and through the primary fluid input manifold. When the primary fluid flows into the primary fluid input cavity 134, optional flow redirection feature 44 splits the primary fluid into two substantially even flows. Each of the flows travels (as indicated by arrows in FIG. 3) over one of the two columns of six reactor core stacks 110. Then the primary fluid traverses the twelve reactor core stacks 110 in parallel, where it gets converted into a primary output fluid, and then exits from the stacks 110 into the primary fluid output cavity 136, where the two separate flows recombine. The primary fluid is then guided through the primary fluid output manifold to the primary fluid output port 245a from where it exits the fluid reactor unit 200. The secondary input fluid enters the fluid reactor unit 200 through the secondary fluid input port 247a which is connected in a sealed manner, to the secondary fluid input manifold (i.e., to through-hole 40 located on the bottom frame piece 14). When the secondary fluid flows into the secondary fluid cavity, it is guided across the multiple reactor core stacks 110 in a manner perpendicular to the primary fluid. The secondary fluid, now converted into a secondary output fluid, then exits the reactor core stacks 110 and enters the secondary fluid output manifold (i.e., through-hole 42 located on the top frame piece 16). From the secondary fluid output manifold, the secondary output fluid travels, in a sealed manner, to the secondary fluid output port 227a from where it exits the fluid reactor unit 200. In embodiments, the primary input fluid is blood. In other embodiments, the primary input fluid is brackish water, salt water, water, or an industrial fluid. In embodiments, at least one secondary fluid is selected from the group comprising oxygen, carbon dioxide, nitrogen, water, water vapor, ethanol, alcohol, alcohol vapor, blood plasma, industrial fluid, and biological fluid. In embodiments, the primary fluid is blood and the two secondary fluids are oxygen and carbon dioxide. In embodiments, the primary fluid is brackish water and the secondary fluid is water vapor.

In embodiments, after a fluid reactor unit 200 is assembled and optionally non-destructively tested, such as described in commonly owned U.S. Provisional Patent Application Ser. No. 62/839,026 (hereinafter “the '026 application” the content of which is hereby incorporated by reference herein in its entirety), the fluid reactor unit 200 is exposed to at least one liquid and at least one subsequent drying process. In embodiments, such liquid exposure and subsequent drying process results in an enhanced blood coagulation reducing surface and/or more biocompatible surface on all of the interior primary fluid contact surfaces of the fluid reactor. In embodiments, such liquid exposure and subsequent drying process ultimately generate an antithrombotic coating where heparin or a heparin substitute material, as known to those skilled in the art and as manufactured by various bio-coating companies, is attached to these primary fluid surfaces, in some cases in such a strong manner that they cannot be easily leached away when exposed to primary fluid flow. One such possible antithrombotic coating is a CARMEDAR BioActive Surface, marketed under the trademark CBAS® Heparin Surface for GORE® Vascular Devices. In embodiments, a fluid reactor unit can undergo multiple liquid exposure and subsequent drying processes.

FIG. 4 depicts an exploded view of a complex fluid reactor unit 300 comprised of a standalone fluid reactor unit housing 302 containing a reactor core 304. The reactor core 304 is shown in a partially exploded view with the last two reactor core assembly parts separated from the rest of the reactor core assembly parts.

The housing 302 includes a lid 306 and a container 308, where the lid 306 and the container 308 each have internal alignment and sealing features 314, thus allowing them to form a fluid tight housing 302. Furthermore, the lid 306 and/or the container 308 can each optionally have at least one external mounting feature 312. Optionally, the container 308 can have at least one internal mechanical alignment feature 316 for mechanically locating the reactor core 304. In embodiments, the container 308 can include at least one weight reduction feature 318 to reduce the overall weight of the housing 302. FIG. 4 depicts the lid 306 with three ports and corresponding fluid lines: (i) a secondary fluid input port 247a and fluid line 247b with an internal O-ring seal 348, (ii) a secondary fluid output port 227a and fluid line 227b, and (iii) a primary fluid input port 225a and fluid line 225b with an internal O-ring seal 326. The internal O-ring seals 326 and 348 are mechanically held, glued or welded into the bottom of container 308 and thus are normally hidden in the view shown FIG. 4. For clarity of teaching, these O-ring seals 326 and 348 are depicted in FIG. 4 with dashed lines. The container 308 has a primary fluid output port 245a and fluid line 245b with an internal O-ring seal 346. In FIG. 4, the fluid lines are shown as barbed hose connectors in a particular spatial arrangement; however, the fluid lines could be made in any other form known to those skilled in the art, and their spatial arrangement can be altered. In embodiments, the fluid lines can be glued or welded to the lid 306 and/or container 308. Optionally, some of the ports can be thresholds to different sized flexible fluid lines, as appropriate for a chosen fluid reactor application, as shown in FIG. 4 with respect to ports and fluid lines 227a,b and 247a,b.

The reactor core 304 shown in FIG. 4 includes a single reactor core assembly which is further made up of multiple reactor core assembly parts, including both reactor core components 100 and reactor core accessories. The reactor core component 100 depicted in FIG. 4 contains multiple reactor core stacks 110 with a width to length ratio of about 1:6. In FIG. 4, two distinguishable reactor core accessories are a primary fluid input endplate 354 and a primary fluid output endplate 352. Such endplates 352 and 354 are sealed and glued to their neighboring reactor core assembly part with optional external sealing grooves 56 and optional holes 58 (as discussed above in relation to FIG. 1). The optional external sealing grooves 56 and holes 58 are used to create a fluid tight seal between neighboring reactor core assembly parts wherever such a seal is needed to isolate and/or complete any primary and/or secondary fluid manifold. As discussed above, the reactor core frame 10 of a reactor core component 100 can be either a single piece or two or more pieces. FIG. 4 depicts an embodiment where the reactor core frame 10 of reactor core subcomponent 100 is a single piece, for example a 3D printed part. In embodiments, O-rings 326 and/or 348 can be located on the endplate 354, rather than lid 306, with a suitable O-ring groove. In embodiments, O-ring 346 can be located on the endplate 352, rather than container 308, with a suitable O-ring groove. In embodiments, the first and/or last assembly parts of a reactor core assembly can be some other reactor core accessory or a reactor core component. In embodiments, the first and last assembly parts can be different from one another.

In embodiments, each of the fluid lines 225b, 245b, 227b, and/or 247b may utilize O-rings to create a fluid tight seal. When present, such O-ring seals may help to accommodate some mechanical height variations of a reactor core 304 and the internal dimensions of a housing 302, thus providing more manufacturing flexibilities and yield improvement capabilities. In embodiments, other means of creating a fluid tight seal may be used, such as a UV cured glue, or other means as discussed above and/or known to those skilled in the art. UV cured glues, or other similar means of creating seals, may be applied whether or not external sealing grooves 56 and holes 58 exist.

In the embodiment shown in FIG. 4, only fluid line 227b, corresponding to the secondary fluid output port 227a, is not sealed with an O-ring seal. Such a seal, however, is not necessary because (i) the housing 302 provides a fluid tight seal and (ii) all of the other fluid lines (225b, 245b, and 227b) have fluid tight seals; therefore, the secondary output fluid is effectively prevented from entering any other fluid manifold or fluid port except that of the secondary fluid output manifold which connects to the secondary fluid output port 227. Thus, the secondary fluid output port 227 and the secondary fluid output manifold are effectively sealed after the lid 306 is sealed to the container 308.

In embodiments, the first and last reactor core components 100 of a reactor core assembly are different from the rest of the reactor core components 304 contained in the assembly in the sense that some of the through-holes 36, 38, 40 and 42 that are present in the other reactor core components are missing from at least one side of the frame 10 of these first and last reactor core components 100. In embodiments, through-holes 36 and/or 40 are not present on one side of the frame 10. In embodiments, through-holes 38 and/or 42 are not present on one side of the frame 10. Omitting through-holes 36 and/or 38 from the first and/or last reactor core components 100 can help prevent the creation of dead flow zones and thus reduce potential coagulation regions, as discussed above in relationship to FIG. 3. In embodiments, through-hole 42 can be located horizontally across from through-hole 40 (and thus, would not be visible in FIG. 4). In embodiments, an endplate 354 and its neighboring reactor core component 100 can optionally have through-hole 42. In embodiments, an endplate 352 and its neighboring reactor core component 100 can optionally have through-hole 40. Such additional through-holes 42 can be beneficial for applications where the secondary fluid output manifold is larger in cross-sectional area than its input manifold, such as when a portion of the secondary fluid output manifold includes the gap area between the reactor core 304 and the inner walls of the container 308. This additional cross-sectional area for the secondary fluid output manifold helps to lower the flow resistance in said manifold. FIG. 4 shows an embodiment where the through-holes 36, 38, 40 and 42, where available, of a reactor core assembly part line up with its matching through-hole 36, 38, 40, or 42 of the other reactor core assembly parts to form their respective manifolds, along with their respective primary or secondary fluid cavities 134, 136, and 120 and fluid lines 225b, 245b, 247b, and 227b. The manifolds directly connect to their respective input or output ports. These manifolds feed into each reactor core component 100, such that, for example, the primary fluid input manifold feeds primary fluid through each primary fluid input cavity 134 thus allowing the primary fluid to traverse the reactor core stacks 110 contained within each reactor core component 100.

In fluid reactor embodiments with multiple reactor core components, the primary fluid output cavity 136 of at least one of the reactor core components may be connected in series with the primary fluid input cavity 134 of a neighboring reactor core component, thereby forming a general primary fluid cavity. In such embodiments, at least one of through-holes 36 or 38 may not be included on such serially connected reactor core components. Such a serial connection allows the primary fluid exiting one reactor core component to at least somewhat spatially equalize its compositional concentrations before entering a downstream reactor core component.

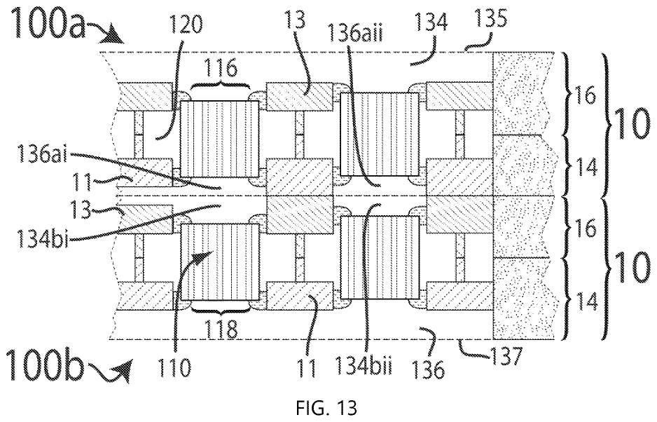

In embodiments with multiple reactor core components, neighboring reactor core components may be designed such that each reactor core stack 110 of one reactor core component is in series with a reactor core stack 110 in its neighboring reactor core component. FIG. 13 depicts a partial cross-section view of two reactor core components 100a and 100b, wherein the exterior surface of a portion of bottom window frame 11 of reactor core component 100a is level with the exterior surface of its bottom frame piece 14 and the exterior surface of a portion of top window frame 13 of reactor core component 100b is level with the exterior surface of its top frame piece 16. This design isolates the primary output fluid of each reactor core stack 110 in reactor core component 100a and allows the fluid to flow serially from a reactor core stack 110 in reactor core component 100a directly into a reactor core stack 110 in reactor core component 100b by flowing directly from primary output fluid cavity 136aii to primary input fluid cavity 134bii. In embodiments, such as depicted in FIG. 13, the frames 10 for at least two neighboring reactor core components 100 are designed such that only some of the reactor core stacks 110 in reactor core component 100a are individually serially connected with downstream reactor core stacks 110 in reactor core component 100b. In other embodiments, the frames 10 for at least two neighboring reactor core components 100 are designed such that all of the reactor core stacks 110 in reactor core component 100a are individually serially connected with downstream reactor core stacks 110 in reactor core component 100b. In embodiments, the frames 10 for at least two neighboring reactor core components 100 are designed such that the primary output fluid from at least one reactor core stack 110 in reactor core component 100a is not isolated from the primary output fluid from at least one other reactor core stack 110 in reactor core component 100a. In such embodiments, as depicted in FIG. 13, the primary output fluid cavity 136ai has a fluid connection to other primary output fluid cavities in reactor core component 100a, thus enabling the primary output fluid of multiple reactor core stacks 110 in reactor core component 100a to combine and enter multiple primary input fluid cavities in reactor core component 100b, such as primary input fluid cavity 134bi. Thus, the primary fluid is able to parallelly distribute between multiple reactor core stacks 110 in reactor core component 100b. In embodiments, the frames 10 for at least two neighboring reactor core components 100 are designed such that the primary output fluid from all of the reactor core stacks 110 in reactor core component 100a is not isolated from any other reactor core stacks 110 in reactor core component 100a, thus enabling a parallel distribution of the primary fluid into the reactor core stacks 110 of reactor core component 100b. In other embodiments, where at least some reactor core stacks 110 in reactor core component 100a are not isolated from at least some other reactor core stacks 110 in reactor core component 100a, the shared primary output fluid cavity 136 of such reactor core stacks 110 is closed off from the primary input fluid cavity 134 of the next reactor core component 100. In some such embodiments, the pooled primary output fluid located in the primary output fluid cavity 136 flows to a through-hole 38 so it can be directed to exit the fluid reactor. In other such embodiments, the primary output fluid is instead directed to the primary input fluid cavity 134 of another reactor core component 100, where such fluid will then flow parallelly, serially, or in a combination thereof through another set of reactor core stacks 110.

In embodiments, the portion of a fluid manifold formed by the connection of matching through-holes 36, 38, 40, or 42 is filled with a complex 3D shaped flow splitter, shear stress reducer and/or flow redirection device (collectively, “internal manifold features”; not shown in FIG. 4). In embodiments, internal manifold features can be inserted after a reactor core assembly is formed. In embodiments, internal manifold features can be built into the through-holes 36, 38, 40, and/or 42 when a reactor core assembly part is manufactured. In embodiments, internal manifold features are injection molded, 3D printed, or made with other manufacturing processes known to the skilled in the art. In embodiments, internal manifold features improve the even splitting of a fluid flow into the various sub-flows that enter fluid cavities 120, 134, or 136. In embodiments, internal manifold features reduce the shear stress for the fluid flow and/or the dead ended flow regions.

In embodiments, a sufficient number (n_{RCS}) of reactor core stacks are arranged in series to achieve a desired minimum secondary fluid transfer rate up to a targeted serially arranged reactor core stack primary fluid flow rate (F_{SRCS}) given the type of reactor core element(s) incorporated therein (i.e., given the width, length, height, fluid channel size and pattern layout, edge zone width, etc. of the reactor core element(s)). Each reactor core stack contained within the serial arrangement includes n_{RCE} reactor core element(s). The reactor core stacks can be serially arranged in variety of ways, including but not limited to, (i) a serial arrangement of reactor core stacks within a single reactor core component; (ii) a serial arrangement of reactor core stacks across multiple reactor core components; (iii) a serial arrangement of reactor core stacks across multiple reactor core assemblies; or (iv) a combination thereof. In embodiments, the total number of reactor core elements (t_{SRCE}, where t_{SRCE}=N_{RCS}*N_{RCE}) contained with the serially arranged reactor core stacks is sufficient to achieve a targeted change in a dissolved secondary fluid concentration (e.g., dissolved gas concentration) of a primary input fluid when it converts to a primary output fluid up to a targeted flow rate F_{SRCS}. In embodiments with multiple separate arrangements of serially arranged reactor stacks, each such arrangement can be connected in parallel, where m_{PSRCS} is the number of serially arranged reactor core stacks connected in parallel. In embodiments, the number of parallelly connected serially arranged reactor core stacks m_{PSRCS} is sufficient to achieve a targeted change in a dissolved secondary fluid concentration (e.g., dissolved gas concentration) of a primary input fluid when it converts to a primary output fluid up to a targeted reactor core flow rate (F_{RC}). The ratio m_{PSRCS}=F_{RC}*F_{SRCS} is the minimum number of arrangements of serially arranged reactor core stacks that need to be connected in parallel in order to achieve a desired reactor core flow rate F_{RC}.

In embodiments where a fluid reactor is an oxygenator incorporating one type of reactor core elements, t_{SRCE} is sufficient to provide the venous blood of a person using the oxygenator with sufficient oxygen in-transfer (e.g., outgoing blood oxygen saturation is >95%) and carbon dioxide gas out-transfer (e.g., a partial pressure drop of outgoing blood is up to 5 mmHg or down to approximately 40 mmHg) to create arterial blood up to a targeted serially arranged reactor core stack blood flow rate F_{SRCS}. In fluid reactor embodiments containing m_{PSRCS} parallelly connected serially arranged reactor core stacks, the targeted serially arranged reactor core stack blood flow rate F_{SRCS} is chosen so that at least one other application-dependent targeted performance parameter is not exceeded. For example, in embodiments, the targeted flow rate F_{SRCS} is selected such that a maximum pressure drop for a specific application is not exceeded. In embodiments where blood is the primary fluid, such a maximum pressure drop can be ≤250 mmHg, ≤150 mmHg, ≤120 mmHg, ≤60 mmHg, or ≤25 mmHg. Examples of performance parameters for a given fluid reactor application that can constrain and/or help determine a targeted serially arranged reactor core stack flow rate F_{SRCS} include, but are not limited to (i) pressure drop, (ii) primary fluid contact surface, (iii) priming volume, (iv) expected use time of a fluid reactor, (v) secondary fluid composition, (vi) primary input fluid composition, and (vii) primary output fluid composition. It should be understood by one skilled in the art that the above formulas and examples regarding fluid flow rate are not representative of all of the various arrangements of the reactor core elements contained within a reactor core. The above is presented to provide one skilled in the art with the knowledge base to determine the number of reactor core elements needed to achieve desired fluid parameters given a particular arrangement of the various components within a reactor core.

FIG. 5 shows a reactor core accessory in the form of a heat exchanger plate 400. In embodiments, a reactor core assembly contains only one heat exchanger plate 400. In other embodiments, a reactor core assembly contains multiple heat exchanger plates 400 that function together to operate as one higher capacity heat exchanger. In some embodiments with multiple heat exchanger plates 400, the build of a reactor core assembly alternates between plates 400 and matching reactor core components 100. In embodiments, the two outer reactor core assembly parts of a reactor core assembly are heat exchanger plates 400, which may help improve the heat transfer capacity for a given reactor core volume while at the same time keeping the primary fluid volume low.

In embodiments, a heat exchanger plate 400 includes a symmetric pair of a left half plate 402 and a right half plate 404, as shown in FIG. 5. In embodiments, a half plate 402 or 404 has a recessed area 406 that is divided by ridges 408 to form an elongated fluid path for a liquid heat exchange fluid and the other half plate 402 or 404 has no recessed area 406. In embodiments, both half plates 402 and 404 have a similar recessed area 406 and matching ridges 408. Optional internal alignment features 18 and 19 can facilitate the mechanical alignment between the half plates 402 and 404 and optional external alignment features 18 and 19 can facilitate the alignment of a heat exchanger plate 400 to a neighboring reactor core assembly part or a part of a fluid reactor unit housing 302. Sealing surface 414 and the top of the ridges 408 can be used to seal the two half plates 402 and 404 together, thus forming an internal heat exchange fluid path that is in thermal contact with the external surface 420 of the heat exchanger 400. The external surface 420 undergoes a heat exchange with the heat exchange liquid and thereby indirectly influences the temperature of any fluid contacting external surface 420, such as a primary fluid of a neighboring reactor core component 100. Not shown in FIG. 5, the recesses 406 of each half plate 402 and/or 404 may optionally have ridges and/or flow direction features that can improve fluid mixing and/or flow rotation of the heat exchange fluid.

As depicted in FIG. 5, a heat exchange input fluid can enter a heat exchanger plate 400 (and, thus, the heat exchange fluid path) through a through-hole 432 and a heat exchange output fluid can exit a heat exchanger plate 400 (and, thus, the heat exchange fluid path) through a through-hole 434. Where a reactor core assembly contains multiple heat exchanger plates 400, the heat exchanger plates 400 can be arranged in parallel or in series. For a parallel heat exchange arrangement, through-holes 432 and 434 are located on both half plates 402 and 404 (as shown in FIG. 5). For a serial heat exchange arrangement, each through-hole 432 or 434 is located on only one half plate 402 or 404. Where a reactor core assembly contains only one heat exchanger plate 400, each through-hole 432 or 434 is located on only one half plate 402 or 404.

In embodiments, a more intricate fluid path may be constructed by, for example, installing a thin flat plate with one or two through-holes between plates 402 and 404. Depending on the quantity and locations of these through-holes, either a serial or parallel fluid flow is obtained in two cavities formed on either side of such a middle plane. In embodiments, other methods known to those skilled in the art can be used to construct a more intricate fluid path.

FIG. 5 depicts a heat exchanger plate 400 with through-holes 36, 38, 40, and 42, where such through-holes become part of the appropriate fluid manifold when the heat exchanger plate 400 is sealed in a reactor core assembly. The sealing area 414 is shaped such that through-holes 36, 38, 40 and 42 are isolated from each other and from the heat exchange liquid. In embodiments, the through-hole layout of the heat exchanger plate 400 is matched to the other reactor core assembly parts of a reactor core assembly in order to ensure continuity of the reactor core assembly's fluid paths. Furthermore, such through-hole matching helps achieve a serial arrangement, a parallel arrangement, and/or a combination thereof of the heat exchange fluid flow path through multiple heat exchanger plates 400, when available.

In embodiments, the heat exchanger half plates 402 and 404 are manufactured from either a metal or a polymer that is compatible with the intended fluid reactor application (e.g., corrosion-resistant, fouling resistant, cleaning chemicals compatible, etc.) and is suitable for operation at the temperature and chemistry of the heat exchange fluid and the primary and secondary fluids. Cleaning fluids may be applied from time to time to remove build ups and/or scale (for example, muriatic acid, acetic acid, or citric acid for calcium-based scale removal). In embodiments, corrosion reducing or bio-film growth suppressant additives are added to the heat exchange fluid to delay and/or prevent clogging events. In embodiments, polymer parts of a suitable polymer material are manufactured by injection molding, stamping, deforming, and/or other manufacturing processes as known to those skilled in the art. For example, such parts can be injection molded from a polycarbonate or 3D printed with a fluid tight part 3D printing process (e.g., SLA, etc.). In embodiments, the primary fluid through-holes 36 and 38 are coated with a functional coating that minimizes buildup of solid (e.g., antithrombotic coatings, etc.) and/or sterilized, using any method known to those skilled in the art, after a respective fluid reactor has been assembled. In embodiments, each half plate 402 and 404 is less than or about 5 mm thick. In embodiments, the thickness between external surface 420 and its corresponding internal heat exchanger fluid contact surface of a half plate 402 or 404 is less than or about 0.5-1.0 mm. In embodiments, recessed area 406 includes suitable stiffening and/or fluid mixing, fluid rotation or turbulence generator ribs, in addition to ridges 408, to improve the heat transfer efficiency from a heat exchange fluid to a primary fluid.

In another embodiment, the heat exchanger plate 400 is made as a single part, for example, 3D printed or molded. In embodiments, the heat exchange fluid path is formed by subsequently removing material from the manufactured part. In embodiments, the thickness between external surface 420 and its corresponding internal heat exchanger fluid contact surface, the heat exchange fluid volume, and the heat exchange fluid path length, are chosen based on the desired fluid reactor performance for a targeted fluid reactor application. In embodiments, material for a heat exchanger plate 400 is chosen based on the desired maximum heat exchanger fluid temperature and heat exchanger fluid flow rate for a given fluid reactor application.

In fluid reactor embodiments where only a single secondary input or output fluid port 245a or 247a is needed, one of the secondary fluid through-holes 40 or 42 can be eliminated from the heat exchanger plate 400. In embodiments of a complex fluid reactor unit 300 having a standalone fluid reactor housing 202 and only a single secondary fluid port 245a or 247a (3-port fluid reactor case), optionally both through-holes 40 and 42 can be eliminated from the heat exchanger plate 400. In such embodiments, the gap between the reactor core 304 and the inside walls of the container 306 form, at a minimum, a portion of the secondary fluid manifold.

FIG. 6 depicts a reactor core accessory in the form of a heat exchanger plate 450 made of three patterned sheets bonded together: a front heat exchanger sheet 452, a flow redirection sheet 454, and a back heat exchanger sheet 456. In embodiments, sheets 452 and 456 are very thin, e.g., approximately 12 to 250 μm thick. In embodiments, sheets 452 and 456 are made of a metal or polymer material. The thinness and material of the sheets allows for a fast and high rate heat transfer from the heat exchange fluid to the primary fluid touching the external surface 470 of the heat exchanger plate 450. At least a portion of the heat exchanger fluid passing through the through-holes 432 and 434 is redirected to flow inside of the heat exchanger plate 450.

In embodiments, sheets 452 and 456 can be made from polycarbonate material or other polymeric material suitable for the intended fluid temperatures and fluid flow chemical composition. In embodiments, flow redirection sheet 454 is made by heat drawing, injection molding, casting, 3D printing, or other means known to those skilled in the art. In embodiments, the flow redirection sheet is cut from a polymer sheet, a metallic sheet, a ceramic sheet, or a sheet of any other appropriate material known to those skilled in the art. In embodiments, sheets 452, 454, and 456 are bonded together prior to being incorporated into a reactor core assembly. In other embodiments, sheets 452, 454, and 456 incorporated into a reactor core assembly one by one.

In embodiments, a reactor core assembly part neighboring a sheet 452 or 456 may have distributed support structures 460 to help support sheet 452 or 456. In embodiments where the reactor core assembly part neighboring sheet 452 or 456 is a reactor core component 100, as shown in FIG. 6, sheet 452 or 456 acts as a cover 135 and thus completes a primary fluid cavity 134 or 136. Support structures 460 further help to ensure primary fluid flow in the cavities 134 or 136 is not impeded.

In embodiments, one of the through-holes 432 or 434 is missing from all sheets 452, 454, and 456 including a heat exchanger plate 450. In such an embodiment, the flow redirection sheet 454 has a least one side hole allowing the heat exchange fluid to exit a reactor core assembly and flow between the outside of the reactor core and the inner walls of the fluid reactor unit housing 302, as discussed above in relation to FIGS. 4 and 5 and the secondary fluid manifold. In embodiments, such as in FIG. 6, flow redirection sheet 454 has a suitable gap or gaps, formed by ridges 408, for flow of heat exchange fluid. Heat exchange fluid enters and exits these gaps through through-holes 432 and 434 located on at least one of the sheets 452 and 456.

In embodiments, heat exchange fluid lines are attached to the fluid reactor unit housing 302. The threshold of the heat exchange fluid line is the heat exchange fluid port. In other embodiments, heat exchange fluid lines are attached to endplates 224 and/or 246. In other embodiments where the fluid reactor unit housing includes endplates 224 and 246 and the outer edges of any reactor core assembly parts, heat exchange fluid lines are attached directly to the sides of the reactor core.

FIG. 7 shows a reactor core accessory in the form of an electrically-powered heater plate 500. In embodiments, a reactor core assembly contains only one electrically-powered heater plate 500. In other embodiments, a reactor core assembly contains multiple heater plates 500 that function together to operate as one higher capacity heater plate system. In some embodiments with multiple heater plates 500, every one or two reactor core components 100 are sandwiched between two heater plates 500. In some embodiments, the build of the reactor core assembly alternates between heater plates 500 and reactor core components 100.

In embodiments, a heater plate 500 includes a cover plate 502 and an electric plate 504 with an electrically powered heating path 506, as shown in FIG. 7. Cover plate 502 is in intimate thermal contact with heating path 506 after cover plate 502 and electric plate 504 are connected in a sealed manner. FIG. 7 depicts two different connector styles for the heating path 506: (i) an end connector 507 and (ii) a pin connector 508 that can penetrate the cover plate 502 through an optional through-hole 509. Optionally, pin connector 508 can extend beyond cover plate 502 and penetrate other reactor core assembly parts and/or a portion of the fluid reactor unit housing 302. In such embodiments, the pin connector 508 connects to a matching connector plug. In embodiments, multiple end connectors 507 from different heater plates 500 are connected to each other in parallel, series, and/or a combination thereof in order to achieve a suitable voltage drop for a given power source. In embodiments, the connections between multiple end connectors 507 are made inside a portion of a fluid reactor unit housing 302 with a suitable fluid-sealed feed-through connection in the respective lid 306 and/or container 308. In embodiments, any other suitable connectors known to those skilled in the art may be utilized in the manufacture of a heater plate 500.

FIG. 7 depicts a heater plate 500 with through-holes 36, 38, 40, and 42, where such through-holes become part of the appropriate fluid manifold when the heater plate 500 is sealed in a reactor core assembly. The sealing area 514 is shaped such that through-holes 36, 38, 40 and 42 are fluid isolated from each other. In embodiments, all through-holes 36, 38, 40, and 42 are optional except for one of through-hole 36 or 38. Optional internal alignment features 18 and 19 can facilitate the correct alignment of the plates 502 and 504. Optional external alignment features 18 and 19 can facilitate the alignment of a heater plate 500 to a neighboring reactor core assembly part or a part of a fluid reactor unit housing 302.

When current is flowing into the resistive heater path, heat is generated and transferred to the external surface 520 of the heater plate 500 through thermal conduction. When a reactor core component 100 is sandwiched, in a sealed manner, between two heat exchanger plates 400, two heat exchanger plates 450, two heater plates 500, or a combination thereof, the primary fluid input cavity 134 and primary fluid output cavity 136 are formed and the imparted temperatures of the external surfaces 420, 470, or 520 change the temperature of any liquid flowing in the primary fluid cavities 134 and 136.

In embodiments, the plates 502 and 504 are manufactured from an electrically insulated material, such as a ceramic, glass, or a polymer sheet, that can handle the imparted temperature necessary for a specific fluid reactor application. In embodiments meant for a blood oxygenation application, the material of the plates 502 and 504 can handle at least about 42° C. In embodiments meant for certain water-based membrane distillation applications, the material of the plates 502 and 504 can handle at least about 60-120° C., depending on scaling problems with the brackish or ocean source water. In embodiments, the plates 502 and 504 are metallic and the heating path 506 is plasma sprayed or printed onto electrical plate 504 over an electrically insulating thin film to prevent it from shorting to the metallic heater material. Such electrically insulating thin film can be made from a ceramic (e.g., Al₂Oₓ), a polymer (e.g., polyimide thin film applied with an adhesive backing), or any other electrically insulating thin film known to those skilled in the art. In embodiments, the metallic heating path 506 and/or an electrically insulating under layer is thermal sprayed or printed onto a suitably shaped stamped and/or molded plastic part (e.g., polycarbonate part, etc.). In embodiments, heating path 506 is capable of generating heat at a rate of 0.5-50 W/cm².

In embodiments, plates 502 and 504 are made from a polymer that has a sufficiently high glass transition temperature that enables mechanic stability for the intended duration of a fluid reactor application at its maximum heating temperature. In embodiments, the heating path 506 is a nickel alloy wire or a thin printed carbon film path enclosed by plates 502 and 504, where plates 502 and 504 are made from a polymeric material with a suitable molding process. In embodiments, plates 502 and 504 are made from a polycarbonate material or a thin sheet of glass that is laser cut, as needed, to create all required features for the heater plate 500. Examples of suitable glass sheets include a sub millimeter thick Willow® glass sheet or a Gorilla® glass sheet as manufactured by Corning. In embodiments, plates 502 and 504 are made of the same material as a reactor core frame 10.

In embodiments, a metallic patterned thin film deposition method (for example, as provided by CVD MesoScribe Technologies Corporation) is used to create the metallic heating path 506 onto plate 504. In another embodiment, the heating path 506 is stamped from a thin alloy metal sheet or foil of sufficient mechanical stiffness and with a suitably high temperature rating. For example, a heating path 506 can be stamped from a suitable metal foil having a high temperature silicone-based adhesive film backing and then transferred to the inner surface 514 of a plate 504. In embodiments, an electrically insulating thin film is first glued to a metallic plate before a heating path 506 is applied. In other embodiments, the heating path 506 is applied with a similar process as is used to manufacture heated car windows. In embodiments, the heating path 506 is created by a selective photo etching process of a metal coated polymer film, such as the process used to make flexible circuits, but tuned to the unique material and heat capacity performance requirements and then stamped or laser cut to the required heating path shape. Other methods of manufacturing thin electrically powered heater plates known to those skilled in the art may be employed.

In embodiments (not shown in FIG. 7), at least one thermal resistor is bonded, printed, or plasma sprayed (or connected in some other manner known to those skilled in the art) onto an insulted plate 504 and respective electrical connections enable the reading of the local temperature of external surface 520. In embodiments, a local temperature feedback signal arising from a temperature sensor imbedded between plate 502 and 504 is used for power control of the heater plate 500.

In embodiments, the through-hole layout of a heater plate 500 is matched to the other reactor core assembly parts of a reactor core assembly in order to ensure continuity of the reactor core assembly's fluid path.

In other embodiments, heater plate 500 is made as a single part, for example, 3D printed or molded. In embodiments, a 3D printer head system that is capable of emitting different materials one at a time or simultaneously, as needed, is used to 3D print the electrical heat path 506 and/or thermal resistor onto the heater plate 500 in the same batch operation as half plates 502 and 504 are 3D printed.

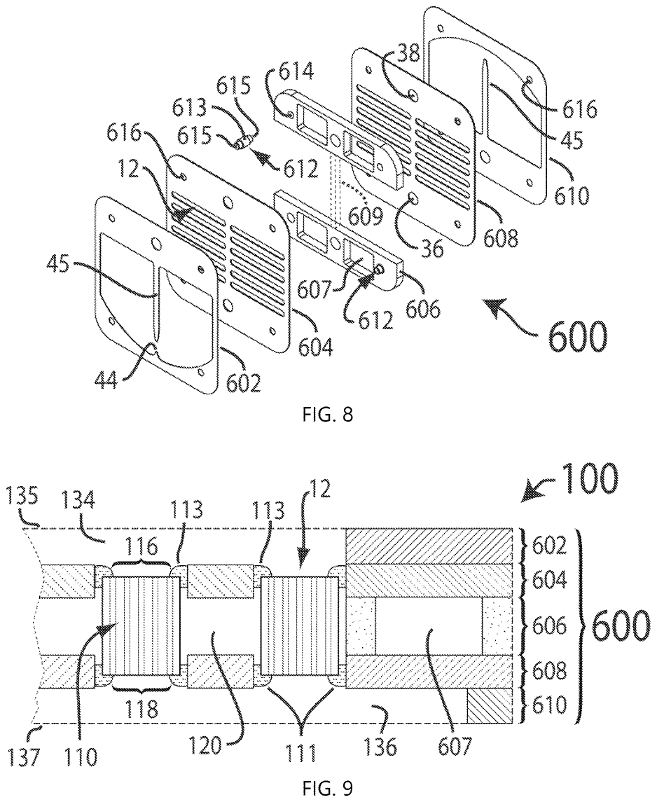

FIG. 8 shows an embodiment where a reactor core frame 600 includes multiple parts made from a flat sheet material. The parts are bonded together in a sealed manner, thus resulting in a single part (i.e., frame 600) with the desired sealed fluid paths. In embodiments, the multiple parts are cut from sheets using any technique known to those skilled in the art, such as stamping, laser or water jet cutting, etc. In embodiments, the sheets can be foils. FIG. 8 depicts a reactor core frame 600 intended for a three-port fluid reactor application, i.e., where only one of a secondary fluid input and a secondary fluid output port exists. In embodiments, reactor core frame 600 can be adopted for a four-port fluid reactor application.

As depicted in FIG. 8, in embodiments, a frame 600 includes (i) a primary fluid input manifold sheet 602, (ii) an input frame sheet 604, (iii) a pair of spacers 606 forming a secondary fluid manifold with optional weight reduction features 607, (iv) an output frame sheet 608, (v) and a primary fluid output manifold sheet 610. In embodiments, frame 600 can have at least two alignment posts 612, depicted in FIG. 8 as a cylinder that has a thicker middle section 613 and thinner end sections 615. The middle section 613 of cylinders 612 align with alignment holes 614 located on the spacers 606 and the end sections 615 of cylinders 612 align with at least two alignment holes 616 located on sheets 604, 608, 602, and 610. As shown in FIG. 8 and FIG. 9, sheets 602, 610, 604, and 608 and spacers 606 can optionally have alignment features in the form of through-holes 616, which correlate to previously discussed alignment features 18. Similarly, end sections 615 of the cylinders 612 can extend beyond sheet 602 and 610, thus correlating to previously discussed alignment features 19. Thus, through-holes 616 and end sections 615 can be used as external alignment features for aligning a reactor core component 100 made from this frame 600 with other reactor core assembly parts and/or fluid reactor unit housing parts, as discussed above. In embodiments, optional internal or external alignment features could include pins, holes, edges or any other alignment feature known to those skilled in the art.

The thickness of sheets 602, 610, 604, and 608 and of spacers 606 are chosen based on the desired dimension of a frame 600 for a specific fluid reactor application. In embodiments, alignment cylinders 612 align sheets 602, 610, 604, and 608 and spacers 606. In embodiments, at least two neighboring sheets and/or spacers with at least two inserted alignment cylinders 612 are bonded together in a sealed manner in one process step which is repeated until the frame 600 is completed. In other embodiments, additional process steps can be used to build frame 600. In further embodiments, all sheets and spacers are bonded or welded together in one process step after they have been aligned with each other with at least two cylinders 612 and/or optional external alignment features 34 (not shown in FIG. 8). In embodiments, a UV curable glue is used to bonding the sheets and/or spacers together and the sheets 602, 610, 604, and 608 and spacers 606 are sufficiently UV transmissive to allow UV light to cure the glue. In embodiments, the sheets 602, 610, 604, and 608 and/or spacers 606 are heat welded or ultrasonically welded together to form a reactor core frame 600.

FIG. 8 depicts two spacers 606 with a fully open gap between them. In embodiments, the two spacers 606 are connected by a middle rib 609 (shown in FIG. 8 with dashed lines), thus forming a single spacer 606. In embodiments, spacers 606 extend at least partially along the sides and/or middle section. Middle rib 609 and/or other extensions to spacers 606 provide more mechanical support for sheets 604 and 608, thus making frame 600 more mechanically stable, and provide further alignment between the spacers themselves. In embodiments, auxiliary spacers can optionally be inserted on the sides between two spacers 606 while the reactor core frame 600 is bonded together and/or while reactor core stacks 110 are bonded into windows 12. The auxiliary spacers can be subsequently removed to reduce the pressure drop of the secondary fluid manifold.

FIG. 9 shows a partial cross-sectional view of a multiwindow reactor core component 100 assembled with a frame 600 (as shown in FIG. 8) oriented perpendicular to the longest direction of its windows 12 and parallel to a line connecting the primary fluid output manifold sheet 610 to primary fluid input manifold sheet 602. The height of a primary fluid input manifold sheet 602 defines the height H_{PFIC} of the primary fluid input cavity 134. The height of a primary fluid output manifold sheet 610 defines the height H_{PFOC} of the primary fluid output cavity 136. The thickness of the sheet 604 defines the height T_{twf} and the thickness of the sheet 608 defines the height T_{bwf}. Spacers 606 (including any optional middle rib 609 or other spacer extensions) separate the output frame sheet 608 from the input frame sheet 604, thus setting the height H_{SFC} of the secondary fluid cavity 120. In embodiments, where two similar reactor core components 100 are neighboring reactor core assembly parts, the total height of a cavity 134 or 136 is the sum of H_{PFIC} of one component 100 and H_{PFOC} of the other component 100.

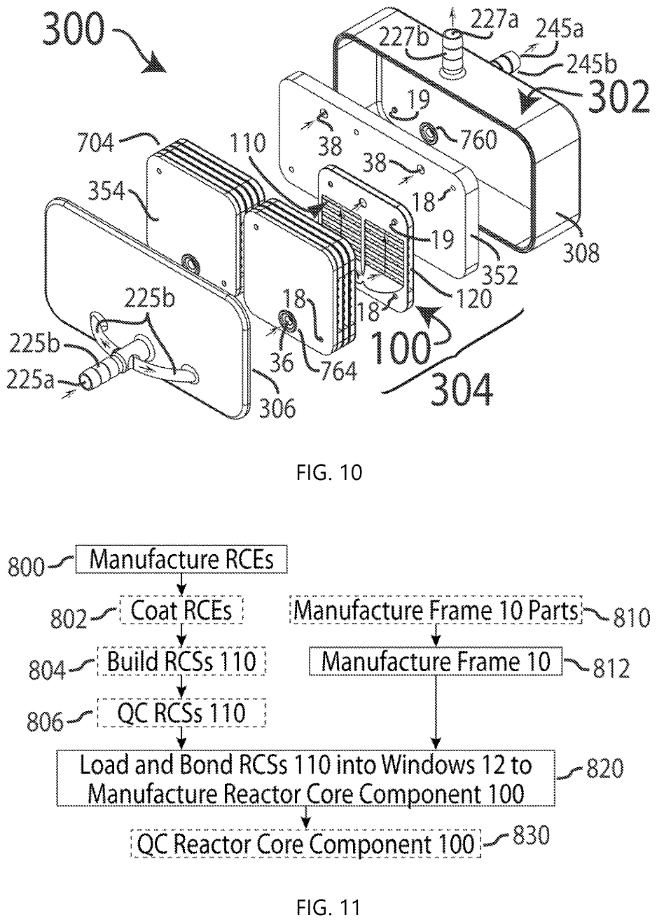

FIG. 10 depicts an exploded view of a complex fluid reactor unit 300 for a 3-port fluid reactor including a standalone fluid reactor unit housing 302 containing a reactor core 304 having two reactor core assemblies 704. The two reactor core assemblies 704 depicted in FIG. 10 share a primary fluid output endplate 352 but have separate primary fluid input endplates 354. In embodiments, reactor core assemblies 704 can share primary fluid input endplates 354 but have separate primary fluid output endplates 352. In other embodiments, reactor core assemblies 704 can have separate primary fluid input endplates 354 and separate primary fluid output endplates 352. In further embodiments, reactor core assemblies 704 can share primary fluid input endplates 354 and primary fluid output endplates 352. In even further embodiments with more than two assemblies 704, some assemblies 704 can share endplates 354 and/or 352 and some assemblies 704 can have separate endplates 354 and/or 352. Optionally, a container 308 can have at least one internal mechanical alignment feature for mechanically locating at least a part the reactor core 304, e.g., endplate 352, as shown in FIG. 10 with the alignment feature 19.

FIG. 10 depicts an embodiment of an endplate 352 with two partial through-holes 38 to collect primary output fluid from each of the reactor core assemblies 704. The primary output fluid is then combined internally in endplate 352 and exits endplate 352 through another partial through-hole (not visible in FIG. 10) that connects to fluid line 245b through an O-ring seal 760 located on the inside of the container 308. FIG. 10 further depicts an embodiment where lid 306 has a primary fluid input port 225a and a bifurcated fluid line 225b that splits the primary input fluid into two, typically equal, separate flow streams that each feed one of the reactor core assemblies 704 through its respective O-ring seal 764.

In FIG. 10, each reactor core assembly 704 includes reactor core components 100, where each reactor core component 100 includes the reactor core frame 10 shown in FIG. 8 bonded with reactor core stacks 110 that have a width to length ratio of about 1:12. Each reactor core component 100 has a secondary fluid cavity 120 that connects to the sidewalls 109 of the reactor core stacks 110. The secondary fluid cavities 120 feed secondary output fluid from the reactor core stacks 100 into the air gaps between the reactor core assemblies 704 and the interior wall of the fluid reactor unit housing 302. The secondary output fluid then exits the housing 302 through fluid line 227b attached to the side walls of the container 308. Three-port fluid reactors, such as the one depicted in FIG. 10, could be used in a membrane distillation application where the secondary fluid is sucked out from the fluid reactor with a vacuum pump. In such embodiments, the secondary fluid could be water vapor that evaporates from a salt water primary fluid. Furthermore, three-port fluid reactors could be used in membrane distillation applications when it is desirable to reduce the energy cost of such a process by decreasing conduction losses.

FIGS. 3, 4, and 9 herein illustrate various fluid reactor embodiments. However, it is to be understood that these depicted embodiments can be further modified by those skilled in the art based on the various concepts, features and aspects described herein without departing from the scope and spirit of this disclosure.

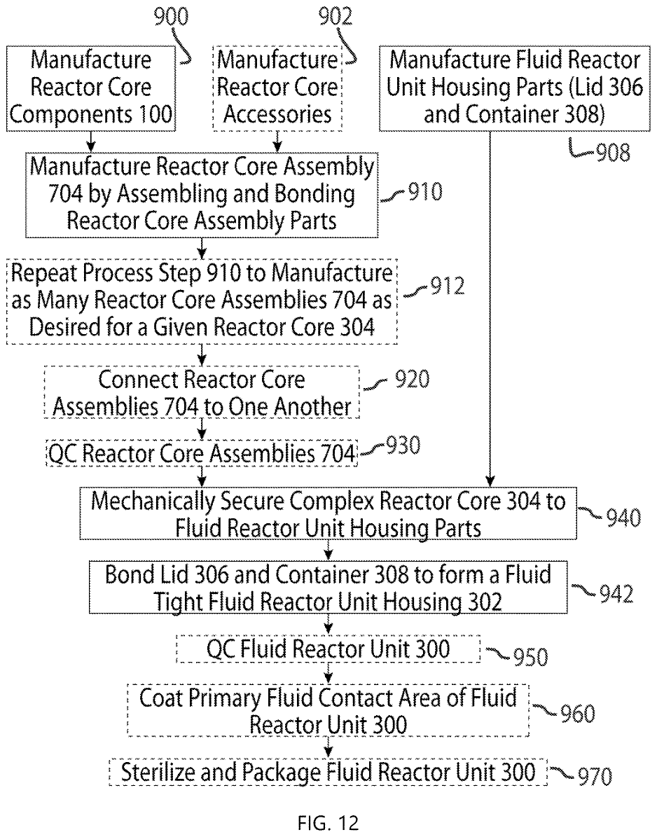

FIGS. 11 and 12 outline manufacturing processes for building reactor core components 100 and complex reactor cores 304, respectively. In FIGS. 11 and 12, it should be understood that process steps outlined with dashed outlines are optional. It is to be understood that additional process steps and/or repeats of the same or similar steps can be added to the processes outlined in FIGS. 11 and 12, as will be appreciated by those skilled in the art reading this disclosure. Furthermore, the order of certain process steps can be rearranged, as will be apparent to those skilled in the art reading this disclosure. The omittance of a process step from FIG. 11 or 12 does not necessarily indicate that such a step is not needed (or optionally includable) for manufacturing reactor core components 100 or reactor cores 304. For example, quality control (QC) steps can be added throughout the manufacturing processes to balance yield and manufacturing costs and/or to provide feedback for maintenance and process improvement opportunities. Additional coating steps and/or other surface modification steps can be added to the manufacturing processes when and where beneficial. Some manufacturing details have already been discussed above and, therefore, hereinbelow are only briefly repeated or outlined to avoid duplications.

FIG. 11 outlines some manufacturing process embodiments for building reactor core components 100. Reactor core components 100 include reactor core elements and reactor core frames 10. Thus, for simplicity, FIG. 11 depicts the manufacturing of reactor core components 100 in two branches that converge when the reactor core elements and reactor core frames 10 are bonded together.

The branch covering reactor core elements begins with process step 800. Process step 800 is the manufacturing of reactor core elements (RCE), which is discussed in detail in the '375 application. Optionally, these reactor core elements can be coated with one or more process steps 802. Some such coating process steps 802 have been discussed hereinabove, as well as in the '375 application, and will be discussed further below. Furthermore, coating process steps 802 may be specifically tailored for a specific application. For example, in fluid reactor embodiments with a water-based primary fluid (e.g., oxygenators, salt or brackish water desalination, etc.), a hydrophobic coating (e.g., commercially available FOTS or FDTS-based coatings by an MVD process or Teflon-based coatings by an i-CVD process) may be applied to the reactor core elements during process step 802.

As discussed in the '375 application, the top and bottom surfaces of a c-VACNT reactor core element have different roughness, with the bottom side typically being much smoother. In embodiments, the bottom and/or top surface of a c-VACNT™ reactor core element is slightly roughed up (e.g., less than about 0.1 to 200 μm deep) prior to coating process step 802. Such a roughing step can cause the top and/or bottom surface to become rougher on a micro to nano level scale. In embodiments, such a roughing step is accomplished (i) in a vibratory polishing table having a felt surface with each reactor core element slightly weighted down in a shallow water pool, as discussed in the '375 application; (ii) by sliding the reactor core element over a fine wet sand paper with a small force applied; (iii) by an O₂ plasma treatment; (iv) by very shallow, laser-based surface ablation with a high speed rastering pattern; or (v) by any other means as known to those skilled in the art. Such a roughing step can help (i) to increase the contact angle CA for a subsequent hydrophobic coating and (ii) to increase the longevity of the contact angle CA (i.e., the longevity of capillary repellent force).

In embodiments, a conformal hydrophobic or hydrophilic, long-lasting coating is applied to reactor core elements during process step 802 by (i) first soaking the reactor core elements in an optionally heated hydrophobic or hydrophilic polymer containing solution in a container, which may be at least partially closed to minimize solvent evaporation until all voids of the open-pore cellular network material of the reactor core elements are filled with the solution, (ii) then removing these filled reactor core elements from the solution and air drying them for at least 1 minute, but typically overnight, (iii) then removing any remaining solvent from the reactor core elements by heating the air dried reactor core elements in an oven to a maximum baking temperature of at least about 10° C. over the boiling point of the solvent, but less than the thermal decomposition temperature of the chosen polymer material, (iv) holding the reactor core elements at this maximum baking temperature for about 1 to about 30 minutes, typical about 5 to about 15 minutes, and (v) finally cooling the reactor core elements down to room temperature.

In embodiments, the polymer solution is a Teflon® AF solution. In embodiments, the Teflon® AF solution can be AF 2400, AF 1600, or AF 1601, as sold by Chemours™, dissolved at a concentration of 1% in suitable solvent, such as Fluorinert™ FC-40 or Opteon™ SF-10. In such embodiments, the maximum baking temperature is less than about 310° C. (typically ≤290° C. for Teflon® AF 2400 or ≤200° C. for Teflon® AF 1600 or AF 1601). Heating the polymer solution during step (i) lowers the solution's viscosity, thus shortening the fluid penetration time and resulting in less fluid drag out upon removal of the soaked rector core element. Such heating is beneficial for a solution of Teflon® AF 2400 in FC-40, though not necessary for Teflon® AF 1600 or AF 1601 in FC-40 or SC-10 solvent.

The thickness of the achieved polymer coating depends, amongst other things, on the surface area of the open-pore cellular network material (i.e., the nanocarbon sponge material for the case of c-VACNT material) and the concentration of polymer material in the solution.

For example, for reactor core elements with about 80-100 nm spacing between vertically aligned carbon nanotube ligaments (as defined in the '375 application) soaked in a solution with a polymer concentration of 1%, the above outlined coating process steps result in an about 0.5-5 nm (typical≈1-2.0 nm) thick conformal coating. This is because the solvent evaporates slowly enough from the void space to allow the dissolved polymer material to precipitate to the nearest surface (i.e., the outer surface of the carbon-coated vertically aligned carbon nanotube ligaments), thus forming another conformal coating over the already carbon-coated surface. This additional conformal coating may be very weakly chemical bonded to the nanocarbon sponge material, if bonded at all. As discussed below, the rougher the surface morphology of the nanocarbon sponge material on a nanometer level prior to applying the thin polymer film coating, the higher the longevity and durability of the thin polymer film coating when exposed to fluids, regardless of whether such fluid exposure is static or dynamic.

Traditional commercial vapor phase coating processes, such as MVD or iCVD, have a depth penetration power problem. These processes typically can only apply a coating inside a channel of width/length≤¼, which results in a thinner and/or missing coating on the inside of the c-VACNT open cellular pore structure while potentially clogging of the pores near the outside of these structures.

On the other hand, the above discussed liquid-based polymer solution coating processes have no such coating thickness uniformity problems and produce substantially conformal coatings throughout 2-4 mm tall c-VACNT structures. In addition, due to the bi-continuous tortuous phase structure of the open-pore cellular network material and the substantially conformal nature coatings resulting from the above discussed liquid coating process, chemical bonding of the polymer material with the surface of the carbon-ligaments is not needed to create a long lasting coating performance, even when the polymer is a polytetrafluoroethylene material that is normally difficult to bond to any material.

Performing a roughing step on the top and bottom surface of a c-VACNT reactor core element prior to the coating process step 802 further improves the adhesion of the polytetrafluoroethylene coatings to a reactor core element, particularly to the previously smooth bottom surface of the reactor core element, since polytetrafluoroethylene coatings have practically no binding force to a flat carbon film present on the bottom surface prior to such roughing step. During one experiment, c-VACNT reactor core elements underwent a coating process step 802, as discussed above, with a Teflon AF 2400 solution, but did not undergo a roughing step prior to step 802. The Teflon AF 2400 coating on the smoother bottom surface of the c-VACNT reactor core element held up (contact angle CA remained>about) 90° for about 21 days when constantly soaked in water, with daily contact angle CA checks, while the rougher top surface held up for over 72 days. The smoothness of the bottom surface reduces the mechanical adherence of the coating over time; therefore, performing a suitable surface roughing step prior to coating process step 802 can increase the lifetime of the bottom surface coating by at least 15-30 times compared to what the lifetime of the coating would have been without such roughing step. Performing a suitable surface roughing step prior to coating process step 802 can increase the lifetime of the top surface coating by at least 5-10 times compared to what the lifetime of the coating would have been without such roughing step. The roughing step increases the lifetime of the bottom surface coating more than the top surface coating because the top surface is rougher than the bottom surface to begin with (i.e., prior to the performance of any roughing step). With a more fully optimized roughing process, both the top and bottom surface coatings held up for over one year.

In embodiments, such as discussed above, coating process step 802 can be done via the above-outlined liquid deposition process, resulting in the formation of a solid conformal polymer thin film. In embodiments, other polymer solutions can be used in the above discussed liquid deposition process to deposit a solid conformal polymer film, provided any temperatures and time frames are adjusted for the specific polymer solution at hand. In embodiments, the thickness of the coating applied during process step 802 is proportional to the concentration of the polymer in the solvent and can thus be easily fine-tuned. In embodiments where the surface area of the open cellular network material is sufficiently known, the average thickness of the coating can be calculated by measuring the weight change before and after the coating process step 802 or by other analytical methods known to those skilled in the art. In embodiments, a coating process step 802 may be performed by any other method known to those skilled in the art. In embodiments, a coating process step 802 applies a coating of a suitable material for a particular fluid application.

In embodiments, a coating process step 802 results in the creation of a thin wall membrane film covering the fluid channels in the reactor core elements. In embodiments, such a membrane film is applied via reaction-based polymer chemistry involving two fluids. One fluid fills any fluid channels while the second fluid fills the void space of the reactor core element's open-pore cellular material. The interaction between the two fluids cause the precipitation of a solid polymer film at their interface. Such precipitate film can have porous structures or selective diffusion properties that are desirable for a given fluid reactor application. In embodiments, a prior coating is applied to either the fluid channels or the open-pore cellular material to ensure that the two fluids do not intermingle. For example, if the whole open-pore cellular material is first treated with a sufficient hydrophobic coating, then during a coating process step 802 involving two fluids, if one fluid is water-based, that fluid will not penetrate the open-pore cellular material, thereby allowing a wider range of reaction chemistry.

In embodiments, multiple reactor core elements can undergo optional process step 804 to be built into suitable reactor core stacks 110, as discussed in the '375 application. In embodiments, during process step 804, at least two reactor core elements are stacked on each other, optionally separated by a suitable thin spacer, and then a side seal is applied between the reactor core elements to create fluid isolation between any existing primary and secondary fluid paths and to mechanically hold the reactor core elements together. Where thin spacers are utilized in building a reactor core stack 110, the thickness of the spacers is at least about equal to the average diameter of fluid channels of the reactor core elements. In embodiments, at least one reactor core element can be stacked onto an already existing reactor core stack 110, thus increasing the fluid processing capacity of the stack 110 at the expense of an increased pressure drop.

As discussed above, hydrophobically coated reactor core elements cannot form a strong chemical bond with sealing materials. Therefore, in embodiments where such hydrophobically coated reactor core elements are used to build a reactor core stack 110, to prevent a seal failure, the coating is sufficiently hydrophobic (i.e., contact angle CA>90° C.) and is applied to reactor core elements that have micro to nano surface roughness, thus providing a sufficiently tight and conformal mechanical seal. A substantially conformal mechanical seal has sufficient mechanical bonding strength such that these reactor core stacks 110 can be mechanically handled and bonded to a frame 10, which then further helps to hold the reactor core stack 110 together.

In embodiments, reactor core stacks 110 can undergo an optional quality control (QC) step 806. In embodiments, QC step 806 involves testing reactor core stacks 110 for leaks via any suitable method(s), such as the methods discussed in the '026 application. In embodiments where QC step 806 is performed via the methods discussed in the '026 application, the order of the components in the testing system described in the '026 application can be rearranged, such as by placing the subsystem before the test unit to prevent a temperature drop of the primary fluid before it arrives at the test unit, thus helping to maintain the primary fluid at the test unit for very low flow speeds. Such adjustments to the methods discussed in the '026 application can be made for any QC step involved in the process of making a fluid reactor.

The branch covering reactor core frames 10 begins with optional process step 810. During optional process step 810, reactor core frame parts are manufactured, as discussed above in relation to FIG. 1 and FIG. 8 or by any other means known to those skilled in art. During process step 812, either a reactor core frame 10 is manufactured as a single piece or a reactor core frame 10 is assembled from the parts made in process step 810. In embodiments, a reactor core frame 10 is manufactured or assembled by any mean known those skilled in the art, such as those discussed above in relation to FIGS. 1, 2, 3, and 7.

In embodiments, a reactor core component 100 is manufactured during process step 820 by loading reactor core stacks 110 into the windows 12 of a frame 10 and bonding the reactor core stacks 110 to the edges of the windows 12 to form a fluid tight mechanical encapsulation, as discussed above. In embodiments, the reactor core stacks 110 can be bonded to the edges of the windows 12 by using a seal, a bond, an isolation method, a weld, a UV curable glue injection, a UV light transmission to cure a UV curable glue behind a sufficient transparent surface, a very local frame material melting and repositioning process, or any other means known to those skilled in the art (hereinafter, all such bonding methods are referred to as a “bond”). In embodiments, reactor core stacks 110 can undergo an optional quality control (QC) step 806. In embodiments, reactor core components 100 can undergo an optional QC step 830, such as discussed in the '026 application.

In embodiments, a simple fluid reactor 200, such as shown in FIG. 3, can be manufactured by (i) manufacturing a reactor core component 100 as outlined in FIG. 11, (ii) manufacturing endplates 224 and 246, and (iii) either (a) bonding all three elements together in one process step or (b) first bonding the endplates 224 or 246 to one end of the reactor core component 100 and then bonding the other endplate 224 or 246 to the other end of the reactor core component 100, as discussed above.

FIG. 12 outlines some manufacturing process embodiments for building a complex fluid reactor 300, such as those shown in FIG. 4 and FIG. 10. Complex fluid reactors 300 include a fluid reactor unit housing 302 containing a reactor core 304. A reactor core 304 includes at least one reactor core component 100 and can optionally include at least one reactor core accessory. Therefore, for simplicity, FIG. 12 depicts the manufacturing of a complex fluid reactor 300 in three branches, two of which converge when a reactor core 304 is built from reactor core components 100 and any optional reactor core accessories and the last of which converges when the reactor 300 is inserted into the fluid reactor unit housing 302.

The first two branches, beginning with process steps 900 and 902, respectively, cover the manufacturing of the reactor core assembly parts. During process step 900, reactor core components 100 are manufactured and optionally quality controlled, typically as outlined in FIG. 11. Process step 902 is the manufacturing of any reactor core accessories that will be incorporated into the reactor core 304, as discussed above. Since reactor core accessories are not necessary for building a reactor core 304, process step 902 is optional; however, for some fluid reactor applications, it is beneficial to incorporate reactor core accessories in the reactor core 304.

In embodiments, a reactor core assembly 704 is manufactured during process step 910 by bonding reactor core assembly parts with one another until the assembly 704 is built to the desired length and functionality. In embodiments, a reactor core assembly 704 is manufactured from only the reactor core components 100 previously manufactured in process step 900. In embodiments, a reactor core assembly is manufactured from only the reactor core accessories previously manufactured in process step 902. In further embodiments, a reactor core assembly 704 is manufactured from a combination of reactor core components 100 and reactor core accessories. In embodiments, a reactor core assembly 704 can include only one reactor core component 100. In embodiments, a reactor core assembly 704 can include only one reactor core accessory. The reactor core assembly parts included in a particular reactor core assembly 704 can be bonded together in any order and by any means appropriate for a given fluid reactor application. In embodiments, at least some of the reactor core assembly parts containing a reactor core assembly 704 can first be assembled together and then undergo a bonding process.

In embodiments, a reactor core assembly can include a single reactor core component 100 and two endplates 352 and 354. During process step 910, the reactor core component 100 can be assembled between the endplates 352 and 354 and then the assembled reactor core assembly parts can undergo a bonding process to form a complex reactor core 304. This arrangement allows, for example, equal UV light exposure from the front and back side of the reactor core assembly when UV light is used during the bonding process step 910.

In embodiments, a reactor core 304 includes a single reactor core assembly 704. In other embodiments, a reactor core 304 includes multiple reactor core assemblies 704. For such embodiments, optional process step 912 is performed, during which process step 910 is repeated as often as needed to build as many reactor core assemblies 704 as are desired for a given reactor core 304. In embodiments, the reactor core assemblies 704 included in a reactor core 304 can be identical. In other embodiments, the reactor core assemblies 704 included in a reactor core 304 can be different from one another. In further embodiments, some reactor core assemblies 704 can be identical to one another while other reactor assemblies 704 can be different from one another.

In embodiments containing multiple reactor core assemblies 704, at least some of the reactor core assemblies can be connected to one another. Such an optional connection is made during process step 920. In embodiments, a reactor core accessory manufactured during process step 902 can be used to connect two reactor core assemblies to one another. In embodiments, process step 920 can involve bonding multiple assemblies 704 to a common endplate 352 or 354, as depicted and described in relation to FIG. 10. In embodiments, a fluid connection line can be used to connect multiple assemblies 704. In embodiments where one of the assemblies 704 is solely made up of reactor core accessories, at least one reactor core accessory of the assembly 704 is connected to at least one reactor core assembly part of a different reactor core assembly 704. Connections between reactor core assemblies 704 can be formed in a serial, parallel or combination thereof manner with sealed fluid lines, electrical connections, and/or by any other means known to those skilled in the art. For example, such connections can be made with an endplate 352 having an internal fluid and/or electrical connection path. In embodiments, process step 920 can be performed after the multiple reactor core assemblies 704 are inserted into a fluid reactor unit housing 302.

Once all reactor core assemblies 704 are completed and optionally connected to one another via process step 920, the complex reactor core 304 can undergo an optional QC step 930, such as discussed in the '026 application. In embodiments, each reactor core assembly can individually undergo QC step 930.

The branch covering a fluid reactor unit housing begins with process step 908. Process step 908 is the manufacturing of a lid 306 and a container 308. Such housing parts can be manufactured, for example, via the same methods used to manufacture a reactor core frame 10 or by any other means known to those skilled in the art. Any fluid lines required for a given fluid reactor unit application can be either built into the housing parts directly or bonded to the housing parts afterwards.

During process step 940, the complex reactor core 304 is mechanically secured, as needed, to the housing parts. In embodiments, process step 940 involves inserting the complex reactor 304 into the container 308 and mechanically securing them to one another. In embodiments, process step 940 involves mechanically securing the complex reactor core 304 to the lid 306. In further embodiments, some of the reactor core assemblies 704 of the reactor 304 are inserted into the container 308 and mechanically secured thereto, as needed, while other reactor assemblies are mechanically secured to the lid 306. In embodiments, such as shown in FIG. 4, optional O-ring seals can be used to seal any available through-holes of the reactor core 304 to its corresponding fluid line attached to the housing parts. In embodiments, connections between multiple reactor core assemblies 704 are applied after the assemblies are appropriately secured to the housing part, but prior to process step 942. Once the complex reactor core 304 is appropriately secured to the housing parts, the lid 306 and container 308 are bonded together during process step 942 to form a fluid tight fluid reactor unit housing 302. In some embodiments, this process step 942 also results in the completion of a fluid reactor unit 300. In other embodiments, the fluid lines may be connected to the housing 302 after process step 942. During process step 942, any remaining internal mechanical connections are also created, such as bonding a reactor core assembly 742 previously connected to the container 308 during process step 940 to lid 306.

A fluid reactor unit 300 can undergo a QC step 950, such as discussed in the '026 application or using any other technique known to those skilled in the art, following process step 942 and/or following any further processing. In embodiments, a fluid reactor unit 300 can undergo optional process step 960 to coat its primary fluid contact areas. For example, during process step 960, a fluid reactor unit's primary fluid contact areas can be coated with an antithrombotic coating applied by one or more liquid exposure/drying step cycle, as discussed above. In embodiments, a fluid reactor unit 300 is sterilized during optional process step 970. In embodiments, process step 970 is done by exposing a fluid reactor unit 300 to an ethylene oxide gas and subsequently packaging the fluid reactor unit in a sterile box until use. Process step 970 is beneficial for certain fluid reactor applications, such as oxygenator applications.

In embodiments, a fluid reactor can be used as an oxygenator. In embodiments, both the blood pump and the gas tanks used for the sweep gas (i.e., secondary input fluid) of the oxygenator are absent, thus creating a portable artificial lung with maximum mobility. In embodiments, if needed, electrical heaters 500 can be used to warm the blood of a portable artificial lung. A portable artificial lung may also be referred to as a wearable artificial lung, such as when the artificial lung is incorporated into a vest system. In embodiments, a portable artificial lung can include a battery pack to (i) provide as needed power for an air pump to generate the as needed sweep gas flow rate, (ii) warm the blood with electrical heater, and/or (iii) send sensor data signals to a recording/warning system. In other embodiments, an O₂ tank is used to provide a sweep gas and the portable artificial lung is pumped by the human heart, thus providing a smaller priming volume PV, lower blood contact surface area SZER, and a sufficiently low pressure drop so as to enable its operation without the use of an external pump. Lowering the priming volume, blood contact surface area, and/or pressure drop can reduce damage to the circulating blood. Therefore, a trade off can be made between a portable oxygenator system with maximum mobility (i.e., minimal cords/hoses attached) and a portable oxygenator system with a gas tank on wheels having a smaller pressure drop and/or priming volume PV and membrane surface area SA_{FR} (which is typically the majority of the blood contact area of foreign material) which may provide overall less blood damage or other complications during and/or after a patient is connected to such an oxygenator system.

In embodiments, at least one oxygenator is incorporated into a vest system having a vest worn by a patient and having at least one of (i) a battery pack, (ii) a wireless communication system, (iii) a control system, (iv) a computerized control system, (v) an air pump speed control system, (vi) an electrical power cable, (vii) a retractable electrical power cable, (viii) a sensor, (ix) a temperature sensor, (x) an O₂ blood gas concentration sensor, (xi) a CO₂ blood gas concentration sensor, (xii) a blood flow sensor, (xiii) a blood pressure sensor, (xiv) control software, (xv) alarm functionality, (xvi) alarm communication ability, (xvii) sensor data transmission capability, (xviii) remote programmability, (xix) a battery pack monitor sensor, (xx) a battery pack recharger, (xxi) wireless battery pack recharging capability, (xxii) an air flow rate sensor, (xxiii) an air flow pressure sensor, (xxiv), means to securely mount at least one oxygenator, (xxv) means to switch from one oxygenator to another, (xxvi) means to switch from battery pack operation to connected power cable operation, (xxvii) means to manually provide sufficient air flow, (xxviii) means to inject fluids, (xxix) means to remove blood samples, (xxx) means to prime the oxygenator, (xxxi), means to connect a oxygenator to a body with venovenous (VV), venoarterial (VA), or other types of cannulation, (xxxii) means to inject blood thinner, saline, drugs, or blood products into the blood stream, (xxxiii) means to bypass the oxygenator; (xxxiv) means to switch from one oxygenator to another oxygenator, (xxxv) means to switch from one type of oxygenator to a different type of oxygenator, (xxxvi) means to switch to a backup oxygenator, (xxxvii) means to guide and protect hoses, electrical cords, sensor lines and/or cannulas, (xxxviii) indicators for status of system, (xxxix) an auditable alarm, (xl) a visual alarm, and/or (xli) pulse oximeter. In embodiments, a vest system could have any other additions as deemed appropriate for a given application.

### EXAMPLES

For simplicity, all of the below examples describe the case where all of the reactor core stacks 110 in a reactor core contain a single reactor core element. It is to be understood that these examples can be modified by those skilled in the art reading this disclosure, based on the teachings herein, to include reactor core stacks 110 that contain more than one reactor core element. It should be further understood that these examples can be modified by those skilled in the art reading this disclosure, based on the teachings herein, to arrange the reactor core stacks 110 in a manner most suitable to achieve the desired fluid reactor parameters (e.g., arranging reactor stacks 110 in series and then further arranging such serially arranged reactor core stacks 110 in parallel in order to achieve a targeted secondary fluid transfer rate, serially arranged reactor core stack primary fluid flow rate (F_{SRCS}), and total fluid reactor maximum output flow rate). Similarly, if one reactor core element alone is not able to achieve a targeted minimal secondary fluid transfer rate for a maximum target flow rate F_{SRCS} discussed in the examples below, multiple such similar reactor core elements can be connected in series for effectively a higher capacity reactor core stack to achieve the targeted minimal secondary fluid transfer rate (with a respective pressure drop APFR penalty) and multiple such reactor core stacks can then be connected in parallel to achieve the targeted total fluid reactor maximum output flow rate capability.

### Example 1: Fluid Reactor Incorporating a Single Hydrophobic Reactor Core Element

Multiple fluid reactors were built with each incorporating (i) a machined polycarbonate endplate 224 with three quick connections to ¼″ plastic tubing, where each quick connect port corresponds to either a primary input fluid line 225b, a secondary input fluid line 247b, and a secondary output fluid line 227b; (ii) a machined polycarbonate endplate 246 with one quick connection to ¼″ plastic tubing corresponding to a primary output fluid line 245b; and (iii) a single reactor core component 100 containing a single reactor core element bonded into a single window 12 of a single part reactor core frame 10 that was 3D SLA printed using a Formlabs' Clear Resin material on a Form 2 commercial printing station. For each fluid reactor, three Viton O-rings were imbedded in separate O-ring grooves on endplate 224 and one Viton O-ring was imbedded in an O-ring groove on endplate 246 to seal to a respective reactor core component 100. Each fluid reactor was held together with screws 262 and nuts 268.

The reactor core elements (herein defined as RCE500S reactor core elements) incorporated into the frames 10 were made by modifying premade ST1 samples having (i) a length L≈30 mm, a width W=15 mm (i.e., a width to length ratio of 1:2), a height H_{RCS}≈2 mm; (ii) an exclusion zone width d_{EX}=1.5 mm; and (iii) a 90 degree rotated rectangular arrangement (quasi hexagonal) of round fluid channels having an average diameter OFc≈47 μm with a minimum gap g_{FC}≈19 μm. The ST1 samples (described in and manufactured in accordance with the methods of the '375 application) were cut with a razor blade to create RCE500S reactor core elements with square corners, a width W≈5 mm, and an exclusion zone along its long side of d_{EZ}=0 mm and along its short side of d_{EZ}=1.5 mm. Alternatively, such RCE500S reactor core elements or other reactor core elements can be made directly by using an appropriately designed photolithography mask, as explained in the '375 application.

Prior to inserting each reactor core element into a frame 10, they underwent a coating process step 802 to make them hydrophobic. Multiple reactor core elements were first baked in an H₂ atmosphere for 15 min at 900° C. for purification purposes (using a FirstNano® Easy Tube® 2000 system) and then cooled to room temperature. A quasi-sealed glass enclosure was filled to a>4 mm height level with a commercially available 1% Teflon AF 2400 solution in Fluorinert™ FC-40 solvent (as sold by Chemours) was preheated to ≈50° C. on a hot plate. Preheating helps lower the viscosity of the solution. Multiple H₂-treated reactor core elements were then added to the solution and were soaked for >5 min to ensure that the solution fully wetted the void phase of the open-pore cellular network material of the c-VACNT reactor core element. Afterwards, these soaked reactor core elements were removed from the liquid bath and left to air-dry overnight. The next day, they were placed in a sealed quartz process tube of the same EasyTube® 2000 system and were heated to ≈290° C. under an Ar atmosphere at atmospheric pressure. The reactor core elements were held there for 15 minutes before being cooled to <100° C. Once removed from the system, these reactor core elements were hydrophobic with a contact angle CA≈130-150° on their primary fluid input and output surface. The contact angle was measured with deionized water and with blood serum. Both methods of measuring the contact angle resulted in a similar value for the contact angle, with a≤5-10° difference.

Each of these RCE500S hydrophobic reactor core elements were then bonded into reactor core frames 10, where the window 12 of each frame 10 was≈100-400 μm larger in width and length than that of the reactor core elements. A commercially available hand-held UV curing glue dispensing system with a small stainless-steel needle and a foot pedal actuated, pneumatically controlled dispensing mechanism was used to bond each reactor core element to the top window frame 13 and the bottom window frame 11 of each frame 10. The UV curable glue used for bonding the reactor core elements to the frames 10 is sold by Dymax Corporation under part number 1405M-T-UR-SC. To bond the reactor core element to frame 10, first an approximately ≈600 μm wide bead of the UV curable glue was manually applied into the gap between the inner edge of top window frame 13 and the outer edge of the reactor core element input surface, thus resulting in an uncured race-track shaped corner seal. A UV beam was created by using a commercially available, foot pedal controlled, 405 nm, ≈1 W solid state UV laser whose UV light output was coupled with a lens to a 5 mm diameter liquid light guide to a distal light guide end that was covered with a fiberoptic dental curing tip. The uncured sealing material was then cured by manually guiding a slightly divergent, quasi-uniform UV curing beam along the uncured race-track shaped seal. A fully cured sealing material may be indicated by the color of the glue changing from blue to clear; such color change is visible when the sealing material is no longer under the UV light. After such a seal was made between the top window frame 13 and the outer edge of the reactor core element input surface, the part was flipped over and the same bonding process was applied to between the bottom window frame 11 and the outer edge of the reactor core element output surface, thus completing a reactor core component 100. The resulting top and bottom seals at least partially blocked some of the fluid channels located within approximately d_{SZ}≈200-500 μm from the outer edge. This resulted in a corner sealed sealing zone having on average width d_{SZ}≈300-500 μm, an active fluid channel zone having an available primary fluid input/exit area of CA_{FCZ}≈1.1 cm² that contained N≈30K active fluid channels. The total fluid reactor membrane surface area for active fluid channels (i.e., the sum of all sidewalls of all fully open, active fluid channels) was SA_{FR}=SA_{FCZ}≈0.90 cm². The reactor core embodied by the reactor core component containing one RCE500S reactor core element has an input surface with an active fluid channel zone cross-sectional area of CA_{FCZ}≈1.1 cm². This fluid channel zone also acts as an arterial filter when the primary fluid is blood with a sharp cutoff at the fluid channel diameter OFC.

Next, the integrity of each seal between a frame 10 and its hydrophobic reactor core element was inspected with a microscope. Once the seal was approved, each reactor core component 100 was placed between a set of endplates 224 and 246 and then mechanically tightened together with screws and nuts to compress the 4 O-rings and form a fluid tight seal, thus completing the fluid reactor. As discussed above, the combination of the hydrophobic coating (nm thick film Teflon AF 2400) and the native nano roughness of the c-VACNT open cellular network material structure resulted in a super-hydrophobic contact angle CA>150° at its top (rough) side and a hydrophobic contact angle CA>130° at its bottom (smooth) side. Therefore, aqueous solutions (e.g., water, blood, etc.) can be trapped by capillary repulsion forces inside the fluid channels and, thus, not wet the void phase.

These fluid reactors were tested for being viable oxygenators. Their integrity and performance were tested in accordance with the methods described in the '026 application, but with the control oxygenator relocated between the test device and the liquid flow controller to minimize primary fluid temperature drop at the slowest test flow rates of the test liquid (15-25 mL/min). For these non-destructive tests, the primary input fluid used was deionized water preheated to ≈37° C. with the heat exchanger of the control device. The secondary input fluid for each test device and the control oxygenator was either air, oxygen, nitrogen, or mixtures thereof, depending on which test was performed (oxygenation or de-oxygenation transfer capacity measurement) and if the sweep gas was delivered to the test device or the control device. The control oxygenator was a commercial oxygenator (Medtronic #CB511) with an integrated heat exchanger. Under ISO 7199 test conditions, the control device has a maximum transfer rate of ~420 sccm of O₂ and ≈350 sccm of CO₂ at a maximum blood flow rate rating of 7 L/min and a recommended use time of up to 6 hours. Thus, this control device has, at least initially, a>10X larger oxygenation/deoxygenation capacity than the test device. ISO 7199 test conditions were simulated with water containing an appropriate level of dissolved O₂ (as discussed in the '026 application), which was produced by the control device through the use of an appropriate gas mixture of air, N₂ and/or O₂. A Medtronic #1351 Intersept Cardiotomy reservoir part was used as the primary fluid reservoir. The primary fluid flow rate was regulated with a liquid flow controller (Entegris 6500-T2-F02-H04-M-P2-U1 NT Integrated Flow Controller) and the primary fluid was pumped, at sufficient pressure, through the test circuit with a 640T Medtronic blood pump and pump head (Medtronic BPX-80).

The maximum tested primary fluid flow rate was F_{FCZ}≈150 mL/min and resulted in an initial pressure drop ΔP_{FR}≈20-30 mmHg for different test samples. Lower primary fluid flow rates resulted in an approximately proportionally lower initial pressure drop. This initial pressure drop is higher than the theoretical value ΔP_{FR}≈8 mmHg calculated based on equation (1) in the '375 application or equation (1) below, indicating that the hydrophobic repulsion contributed to the observed pressure drop and/or that possibly not all fluid channels where fully open for the chosen test device.

At a maximum tested primary fluid flow rate of F_{FCZ}=100 mL/min of water and with a sweep gas (i.e., secondary input fluid) flow rate of 50 sccm of O₂, the test device had a dissolved O₂ concentration change from about 22% to >215% and a dissolved N₂ concentration change from 100% to <58%. At a maximum tested primary fluid flow rate of F_{FCZ}=150 mL/min of water and with a sweep gas flow rate of 75 sccm of O₂, the test device had a dissolved O₂ concentration change from about 22% to >180% and a dissolved N₂ concentration change from 100% to <66%.

The decay of the starting O₂/CO₂ transfer capacity for these RCE500S test devices was then monitored over time under different flow rate conditions and different primary fluid input conditions. One fluid reactor's performance was evaluated for over 3 weeks. During this time, the pressure drop slowly increased in a non-linear and non-monotonic manner from ≈30 mm Hg to ≈55 mm Hg. The observed increase in pressure drop was likely due to the clogging of some fluid channels, which may have resulted from chemical leaching out of the coated tubing used in the test circuit and/or algae growth over time due to the non-sterile test setup. After three weeks of testing, the coated Tygon tubing was replaced, but, as the test system was likely already contaminated, within a few days the device related pressure drop APPF increased to over 100 mmHg, at which point the test was terminated. Fluid channel clogging particulates may have also generated from the bearings on the inside of the pump head used, as well from brass fitting used to connect various parts of the test circuit.

Nevertheless, the oxygen and de-oxygen transfer rate capacity enabled by the hydrophobic properties of the coated reactor core reactor elements was very stable, with only a slight decay over time, i.e., ≈10-15% over multiple weeks. Further testing of RCE500S hydrophobically coated reactor core element involved keeping the reactor core element submerged in water for a two-month period. This test resulted in a slow decay from an initial value of CA=156° to CA=139°, i.e., a decrease of ≈11%. Additional contact angle monitoring of coated reactor core elements submerged constantly in water over a 12 month period showed hydrophobic performance is possible for over 12 months when the reactor core elements have undergone a roughing step that creates a surface roughness of at least 0.5-10 μm deep prior to the hydrophobic coating application.

Combining equations (1), (2) and (3) from the '375 application results in the equation (1) below, which describes the relationship between the flow rate F_{FCZ} of a primary fluid having a dynamic viscosity η_{PF} (≈2.78 cP for blood at 37° C., ignoring the Fåhraeus-Lindqvist effect for blood) and the pressure drop APPF across an reactor core element (ignoring hydrophobic repulsion effects) having N active fluid channels inside a sealing zone arranged in a periodic hexagonal layout with an average diameter φ_{FC} and minimal gap g_{FC} between them

$${F}_{FCZ} = N * \frac{Δ {P}_{PF}}{{R}_{PF}} \approx \frac{π * {CA}_{FCZ}}{\sqrt{3} * 64 * {η}_{PF}} * \frac{Δ {P}_{PF} * {ϕ}_{FC}^{4}}{{( {ϕ}_{FC} + {g}_{FC} )}^{2} * h} , \qquad ( 1 ) .$$

For a constant flow rate F_{FCZ}, equation 1 shows that

$$Δ {P}_{PF} ∼ 1 / ( {ϕ}_{FC}^{2} / {( 1 + {g}_{FC} / {ϕ}_{FC} )}^{2} .$$

Equation (1) predicts that, for RCE500S c-VACNT reactor core elements, the pressure drop ΔP_{PF} gets reduced by a factor≈0.6 when the diameter φ_{FC} changes from 47 to 60 μm. Equation (1) further predicts that, for the test device described in this Example, a water flow rate of 150 mL/min results in a pressure drop of ΔP_{PF}≈8 mmHg. However, for test devices that were super-hydrophobically coated, the measured pressure drop for water was ΔP_{PF}≈20-30 mmHg, thus indicating that the hydrophobic repulsion contributed about 12-23 mmHg to the total pressure drop.

For a given reactor core element, the smaller the sealing zone width d_{SZ}, the more fluid channels can be active. Where two reactor core elements have the same primary fluid flow rate per fluid channel, the smaller the reactor core element width W, the higher the secondary fluid transfer rate for each fluid channel. For example, for reactor core elements with a true hexagonal pattern, where the reactor core elements are similar to RCE500S, except that they have rounded corners with a radius=W/2 with W=5.0 mm and d_{SZ}≈0.4 mm, N increases by a factor of ~1.10X for the same diameter φ_{FC}≈47 μm and gap g_{FC}≈19 μm. The reactor core embodied by the reactor core component containing one such reactor core element has an input surface with an active fluid channel zone cross-sectional area CA_{FCZ}≈1.2 cm² which also acts as an arterial filter when the primary fluid is blood. The reactor core embodied by the reactor core component containing one RCE25OR reactor core element entrance area has an input surface with an active fluid channel zone cross-sectional area CA_{FCZ}≈0.6 cm². As with the RCE500S embodiments, these other reactor core element embodiments act as arterial filters when the primary fluid is blood. Furthermore, each reactor core element acts a particle or gas bubble filter with a cutoff at the fluid channel diameter OFC.

For RCE25OR reactor core elements with rotated rectangular or true hexagonal fluid channel layout, which are similar to RCE500S, except that RCE250R has rounded corners with a radius=W/2 with W=2.5 mm and d_{SZ}≈0.25 mm, N decreases by a factor of ~0.5X for the same diameter φ_{FC}≈47 μm and gap grc≈19 μm. The change in the number of active fluid channels N for the reactor core element described above with a radius=W/2 with W=5.0 mm and d_{SZ}≈0.4 mm and RCE250R reactor core elements results in a change in the pressure drop ΔP_{PF} by a similar factor (~1.1⁻¹ X and ≈0.5⁻¹ X, respectively) since each fluid channel's flow rate changes by the same factor for the same total flow rate per reactor core element. In embodiments, the fluid channel pattern layout, the fluid channel diameter φ_{FC}, gap g_{FC}, and/or sealing zone width d_{SZ} of the reactor core element with a width W, length L and height H_{RCS} is adjusted based on the required O₂ gas transfer rate and CO₂ gas removal rate for a maximum nominal blood flow rate F_{FCZ} for an oxygenator application while minimizing (or reducing to an application dependent maximum limit) the maximum pressure drop ΔP_{PF}. Under ISO 7199 test conditions, for 7 L/min of venous blood, the gas transfer rate for commercially available adult oxygenators made with hollow fiber reactor core elements is typically ≈400-450 sccm for O₂ and ≈250-350 sccm for CO₂.

For a reactor core component whose primary fluid cavities 134 and 136 are divided into two split flows, such as due to a flow splitter 45, the pressure drop ΔP_{RFC} across each reactor core element, having non-circular fluid channels and a width W and height H and length L_{RFC}, for a total flow F_{RFC} into or out of a recessed fluid cavity, where the recessed fluid cavity is a primary fluid input cavity or a primary fluid output cavity, can be estimated from the Hagen-Poiseuille's law for non-circular cross section

$$Δ {P}_{RFC} = \frac{{F}_{RFC}}{2} * \frac{12 * {η}_{PF} * {L}_{RFC}}{W * {H}^{3} * ( 1 - 0.63 * \frac{H}{W} )} . \qquad ( 2 )$$

and for primary fluid input or output manifold, excluding any primary fluid input or output cavities, having a length L_{d} and a diameter d the Hagen-Poiseuille's law for circular cross section results predicts a pressure drop ΔP_{d} for a flow rate F_{d}.

$$Δ {P}_{d} = {F}_{d} * \frac{128 * {η}_{PF} * {L}_{d}}{π * {d}^{4}} . \qquad ( 3 )$$

### Example 2: Reactor Core Element with a Low Temperature Applied Hydrophobic Coating

Multiple ST1-like c-VACNT reactor core element samples (where the fluid channels are laid out in a rotated 90 degree rectangular pattern), underwent a roughing process step for their the top and bottom surfaces with either (i) a mechanical process (vibrational polishing on wet felt while being weighted down and subsequently washed in deionized water and/or ethanol solution for 15 min in an ultrasonic bath to remove any lose particles) or (ii) a plasma surface oxidation process step utilizing an air or O₂ plasma at reduced pressure. For example, several samples were exposed at ≈0.5 mmHg to an air plasma at 50 W for 1-10 minutes while resting on a quartz plate. When multiple samples undergo roughing in the same batch process, the samples can be hung from a mounting tree so that the front and back surfaces can be treated at the same time. The tree can be moved (rotationally, linearly, oscillatory, and/or some other motion) through the plasma zone to achieve a more uniform top and bottom surface micro roughening. Subsequent to the (i) mechanical or (ii) plasma surface roughening process, all samples were cleaned in a sonication bath and/or baked in a H₂ atmosphere at 900° C. for 15 min and then cooled down to room temperature to remove, for example, possible hydrocarbon contaminations. These sonication cleaning and/or H₂ baking steps may be optional.

Next, all of the samples were soaked in a closed glass container at room temperature in a 1% solution of Teflon AF 1601 dissolved in SC-10 solvent (available from Chemours). After at least 5 min of soaking, the samples were removed from the solution and air dried overnight. The next day, the samples were split into two groups and baked under an inert Ar gas flow at atmospheric pressure for 15 min above the 110° C. boiling point of the SC-10 solvent. Group A was baked at 200° C., i.e., below the gas transition temperature of Teflon AF 1601 (240-275° C.), and Group B was baked at 290° C., i.e., above the gas transition temperature of Teflon AF 1601. Subsequent testing of these samples showed that, regardless of the roughing process and baking process applied, the resulting polymer coating provided a superhydrophobic surface on both the top and bottom which stayed hydrophobic for more than 12 months of water immersion. These experiments also show that the bakeout temperature does not have to be above the glass transition temperature of the polymer to obtain conformality and durability for this polymer coating process as long as the bakeout temperature is above the boiling point of the solvent at the bakeout temperature and pressure.

In embodiments, a sufficient amount of solvent can be removed from the material at a bakeout temperature that is lower than the boiling point of the solvent at atmospheric pressure. Such embodiments can be accomplished by (i) using a vacuum bake process; (ii) allowing the samples to sit for a sufficiently long period of time in air at atmospheric pressure conditions in an open room or fume hood-like semi-closed off compartment with a constant rate of gas exchange; (iii) flowing inert gas through and around the samples for a sufficiently long time to sufficiently dry out the solvent; (iv) and/or by other means known to those skilled in the art for drying a surface from an absorbed solvent. Electromagnetic energy of any wavelength band or spectral distribution that is sufficiently absorbed by the material (e.g., in the form of IR, visible light, microwave energy, etc.) can also be used to warm up the material to speed up such a solvent surface evaporation/drying process.

### Example 3: Pediatric Oxygenator Design Using Hydrophobic Reactor Core Elements

FIG. 3 shows a design of a fluid reactor incorporating a single reactor core component 100, which, if used as a pediatric oxygenator could incorporate twelve hydrophobically coated reactor core elements, potentially with a total nominal primary input fluid flow capacity of 12*150 mL/min=1.8 L/min where the primary fluid is venous blood warmed to 37° C. To build the reactor core component 100, these reactor core elements can be bonded into a reactor core frame 10. Depending on the specifications of the reactor core elements and the reactor core frame (e.g., length; width; height; exclusion zone; sealing zone; spacing between the top window frame 13 and the bottom side 32 of the top frame piece 16; spacing between the bottom window frame 11 and the top side 30 of the bottom frame piece 14; thickness of top window frame; thickness of bottom window frame; etc.), such a reactor core component 100 could have a secondary fluid cavity H_{SFC}≈1.6 mm, an active number N≈32K of fluid channels inside each sealing zone, and an active membrane surface area SA_{FCZ}≈95 cm². This reactor core component 100 can be built into a fluid reactor where (i) the primary and secondary fluid input and output lines 225b, 245b, 227b, and 247b have an inner diameter of ≈5.3 mm and a length of 20 mm and are sized on their outside for a ¼″ inner diameter plastic blood delivery line, (ii) through-holes 40 and 42 have a diameter ~5.3 mm, (iii) the primary fluid is blood heated to 37° C., (iv) the thickness of the endplates 224 and 246 is≈3 mm, and (iv) the reactor core component 100 is sandwiched between two reactor core accessories in the form of a≈4 mm thick heat exchanger 400 or 450 or electrical heater 500 (not shown in FIG. 3). If the efficiency of each reactor core element is high enough to meet its individual targeted primary fluid flow rate of approximately 150 mL/min, the resulting fluid reactor could have (i) a maximum priming volume PV≈11 mL; (ii) a fluid reactor pressure drop ΔP_{FR} of ≈67 mmHg (ΔP_{FR} can be calculated using equations (1), (2) and (3) and may include an experimental correction for the hydrophobic repulsion effect) for a nominal maximum blood flow rate F_{FR}=F_{RCC}=1.8 L/min (where F_{RCC} is the flow rate for a reactor core component); (iii) an effective total fluid reactor blood contact membrane surface area of SA_{FR}≈12*095=1,140 cm²; and (iv) a projected O₂ transfer rate of >>12*8.6 mL/min≈103 mL/min and CO₂ removal rate of >>12*6. mL/min≈82 mL/min for a secondary input fluid sweep gas flow rate of <0.9 L/min of O₂. The reactor core embodied by the reactor core component containing twelve such reactor core elements could have an input surface with an active fluid channel zone cross-sectional area of ~12*1.2 cm²≈14 cm², where CA_{FCZ}=1.2 cm². The input surface could also act as a particle or gas bubble filter with a cutoff at the fluid channel diameter φ_{FC} and therefore can be used for filtering, blood, particles, clogs, and/or gas bubbles, thus reducing the occurrence of microembolisms in the body. The total heat exchange surface area could be ≈69 cm², unless more heaters 500 or heat exchangers 400 or 450 are being added to reactor core at the cost of some priming volume PV increase.

### Example 4: Pediatric Oxygenator Design Based on Reactor Core Elements with Lower Pressure Drop

When the reactor core elements discussed in EXAMPLE 3 are de-rated to a maximum nominal blood flow of 150/2 mL/min=75 mL/min, two reactor core components 100 (as described in Example 3) are needed to create a maximum nominal flow of 2*12*75 mL/min=1.8 L/min for a pediatric oxygenator. If each reactor core component 100 is sandwiched between two heat exchangers 400 or 450 or electrical heaters 500, the following performance data projections may potentially be obtained: (i) priming volume PV≈21 ml; (ii) total fluid reactor membrane surface area SA_{FR}≈2,280 cm²; (iii) ΔP_{FR} of ≈35 mmHg; and (iv) a projected O₂ transfer capacity of >>24*8.6 ml/min≈206 ml/min and CO₂ removal capacity of >>24*6.8 mL/min≈164 mL/min for a secondary input fluid sweep gas flow rate of <0.9 L/min of O₂. Therefore, at the expense of a factor 1.9X increase in priming volume and 2X membrane surface area SA_{FR}, the pressure drop ΔP_{FR} can be reduced by ≈0.5=1/2 X. The reactor core embodied by two reactor core components each containing twelve such reactor core elements could have an input surface with an active fluid channel zone cross-sectional area of 24*1.2 cm²≈29 cm², where CA_{FCZ}=1.2 cm². The input surface could also act as a particle, gas bubble and/or arterial filter when the primary fluid is blood. The total heat exchange surface area could be ≈138 cm², unless more heaters 500 or heat exchangers 400 or 450 are added to reactor core.

By reducing both (a) the spacing between the top window frame 13 and the bottom side 32 of the top frame piece 16 and (b) spacing between the bottom window frame 11 and the top side 30 of the bottom frame piece 14, the priming volume is reduced while the ΔP_{FR} is increased. According to equation (3), the pressure drop within a primary fluid cavity has 3^{rd} power dependence on the height H_{PFIC} or H_{PFOC} of a primary fluid cavity. By further reducing the height of the bottom and top window frames 11 and 13 of frame 10, the priming volume is reduced without significantly changing the pressure drop ΔP_{FR}.

### Example 5: Adult Oxygenator Design Based on Hydrophobic Reactor Core Elements

FIG. 4 shows a design of a fluid reactor, which, if used as an adult oxygenator could incorporate a total of 12 hydrophobic coated reactor core elements into four reactor core components 100 (as defined in EXAMPLE 3), potentially with an assumed total nominal primary input fluid flow capacity of 4*12*150 mL/min=7.2 L/min, where the primary input fluid is venous blood warmed to 37° C. This fluid reactor can be built such that (i) the primary fluid input and output lines 225b and 245b, and secondary fluid output line 227b have an inner diameter of ~7.4 mm; (ii) the secondary fluid input line 247b has an inner diameter of ≈5.3 mm; (iii) fluid lines 225b, 245b, 227b, and 247b have a length of ≈20 mm and are sized on their outside for a ⅜″ (or ¼″ for the secondary input line) internal diameter plastic blood delivery line; (iv) the primary fluid is blood heated to 37° C.; (v) the thickness of the endplates 352 and 354 is≈3 mm; and (vi) each reactor core component 100 is sandwiched between two reactor core accessories in the form of a ≈4 mm thick heat exchanger 400 or 450 or electrical heater 500 (not shown in FIG. 4). The resulting fluid reactor is expected to have (i) a calculated priming volume PV≈42 mL; (ii) a fluid reactor pressure drop ΔP_{FR} of ≈68 mmHg for a nominal maximum blood flow rate F_{FR}=4*1.8 L/min≈7.2 L/min, where F_{RCC}=1.8 L/min, as discussed above; (iii) an effective total fluid reactor blood contact membrane surface area of SA_{FR}≈48*95 cm²-4,560 cm²; and (iv) a projected O₂ transfer rate of >>48*8.6 mL/min≈412 mL/min and CO₂ removal rate of >>48*6.81 mL/min ~327 mL/min for a secondary input fluid sweep gas flow rate of <3.6 L/min of O₂. The reactor core embodied by four reactor components each containing twelve such reactor core elements could have an input surface with an active fluid channel zone cross-sectional area of 48*1.2 cm²≈57 cm², where CA_{FCZ}=1.2 cm². The input surface could also act as a particle, gas bubble and/or arterial filter when the primary fluid is blood. The total heat exchange surface area could be ≈280 cm², unless more heaters or heat exchangers are added to the reactor core. In embodiment additional heaters and/or heat exchangers 400 or 450 are added to a reactor core to increase its ability to heat and/or cool the primary fluid for a given flow rate, at the expense of some priming volume PV increase.

Again, as discussed in EXAMPLE 4, by changing the construction design parameters, the priming volume and surface area can be traded off against pressure drop ΔP_{FR}. Tightening various manufacturing tolerances can result in a gain in priming volume, while tightening the sealing width d_{SZ} can result in a reduced pressure drop.

As also discussed above, for all pediatric and adult fluid reactor examples with excess O₂ and CO₂ transfer rates, the fluid channel diameter φ_{FC} can be increased until just sufficient O₂ and CO₂ transfer rates are available. For example, such adjustments can be made until O₂ transfer rate is ≈410 mL/min and CO₂ transfer rate is ≈340 mL/min for the total fluid reactor, which then can further reduce the pressure drops of the above fluid reactor designs. In embodiments, the pressure drop is sufficiently low that these fluid reactors can be used without a blood pump, i.e., the blood flow is provided by a human or animal heart alone. This can further reduce the coagulation rate, hemolysis rate, and/or bodily inflammatory responses caused by the prolonged use of such a device. Therefore, this could increase the quality of life of a patient both while connected to such a device and after such connection is removed due to a potentially lower follow-on complication rate.

### Example 6: Adult Oxygenator Using Air as Sweep Gas

When reactor core elements similar to those used in EXAMPLE 3 fluid reactors with their respective assumed gas transfer performance capability are used at a reduced primary fluid flow rate, for example, F_{FCZ}≈25 mL/min and with a respective sweep gas (air) flow rate of 25-150 mL/min, the fluid reactor may have a blood equivalent gas transfer rate of ≈1.5 mL/min for O₂ and gas removal capacity of >>1.2 mL/min for CO₂ for extended use time. Given that the gas transfer rate is at least linear to quadratically related to the width W of the reactor core elements, a reactor core element with half the width W (e.g., 2.5 mm) could have an O₂ gas transfer rate of up to ≈8.6 mL/min and a CO₂ gas removal rate of >>6.8 mL/min for a primary fluid flow rate F_{FCZ}≈2*25 mL/min=50 mL/min and an air flow rate of 50-300 mL/min.

If, for example, sixteen RCE250R parts (L≈30 mm, W≈2.5 mm, 1.25 mm corner radius, H_{RCS}≈2 mm, d_{EX}≈100 μm), are put into a reactor core frame 10 to make a reactor core component 100, the resulting reactor core component may have a nominal maximum primary fluid flow rate F_{RCC} of 16*50 mL/min=800 mL/min. Reducing the sealing width to d_{SZ}≈250 μm could results in N≈16K and SAFc≈0.5 cm² for the same φ_{FC}≈45 μm and g_{FC}=19 μm. Such a reactor core component 100 with (i) 1 mm spacing between the top window frame 13 and the bottom side 32 of the top frame piece 16; (ii) 1 mm spacing between the bottom window frame 11 and the top side 30 of the bottom frame piece 14; and (iii) a 2.5 mm gap between the window 12, could result in (a) L_{PFOC}≈52 mm, (b) L_{PFOC}=49 mm, (c) a total nominal primary input fluid of 16*50 ml/min=0.8 L/min, and (d) a secondary input fluid flow rate of 0.8-2.4 L/min for air as sweep gas. Therefore, nine such reactor core components 100, connected in parallel, may result in an adult oxygenator with a total nominal full-sized adult primary and secondary fluid flow capacity of 7.2 L/min with sufficient O₂/CO₂ transfer level. If, as in EXAMPLE 5, primary and secondary input fluid lines of such a device have an internal diameter of ⅜″, then the device could have (i) a priming volume PV of ≈75 mL, (ii) a pressure drop ΔP_{FR} of ≈30 mmHg for a nominal maximum blood flow rate F_{FR}=9*F_{RCC}=7.2 L/min, and (iii) a total membrane surface area of SA_{FR}≈16*9*0.54.6 cm²≈0.7 m². The total reactor core primary fluid input area could be ≈9*16*0.6 cm²≈84 cm². The fluid reactor could contain a heat exchanger 400 or 450 or electrical heater 400 with a surface area ≈500 cm².

As discussed above, priming volume PV and total membrane surface area SA_{FR} can be traded off against pressure drop ΔP_{FR}. In addition, when excess gas transfer rate capacity exists, the fluid channel diameter φ_{FC} and/or gap g_{FZ} can be changed to get the lowest ΔP_{FR}. In embodiments, the ΔP_{FR} is so low that oxygenators can be pumped only by a human heart in sufficiently good condition, i.e., without needing a blood pump, which then further reduces the blood damage rate (platelet activation, hemolysis, etc.).

Simple heat transfer calculations can be used to determine the power needed to raise the temperature of the primary fluid. For example, in embodiments where the primary fluid is water moving at a flow rate of 7.2 L/min and the total heating area per heater is ≈60 cm² (double sided), to raise the temperature of the water by 10 degrees Celsius, an electrically powered heater 500 with an ability to generate heat at a level of 8-20 W/cm² could be used, depending on any thermal conduction losses. Similarly, if a heat exchanger 400 or 450 is used and the heat exchanger input fluid is 12 L of water that is 10° C. hotter than the primary input fluid, about 8.6 W/cm² of power are available for heat transfer. The more thermally conductive and thinner the walls of the heat exchanger 400 or 450 or heater 500, the more heat will transfer into the primary fluid and the higher the efficiency of the heat exchanger or heater.

### Example 7: Wearable, Pumpless, Air-Consuming Artificial Lung

Reactor core components 100 utilizing RCE25OR reactor core elements may also be used at a reduced primary blood flow, e.g., ≈20.8 mL/min. Such a reactor core component 100 can be built as detailed in EXAMPLE 6 but instead with (i) 2 mm spacing between the top window frame 13 and the bottom side 32 of the top frame piece 16; (ii) 2 mm spacing between the bottom window frame 11 and the top side 30 of the bottom frame piece 14, (iii) a total nominal reactor core component primary input fluid of F_{RCC}=16*20.8 mL/min=332.8 L/min, and (iv) a secondary input fluid flow rate of 333-2,000 mL/min for air as sweep gas. A wearable, blood pumpless, air-consuming artificial lung (PAAL) with a primary and secondary fluid flow capacity of 4 L/min and sufficient O₂/CO₂ transfer for a full-sized adult could possibly be built with twelve of these reactor core components 100 connected in parallel and can have integrated electrical heaters 500. A PAAL device can be attached to a patient via venovenous (VV) or venoarterial (VA) cannulation in partial bypass to a lung and provide respiratory support. Unlike a traditional extra-corporeal membrane oxygenator, a PAAL may not require the use of a blood pump and may use air as the sweep gas. With ⅜″ input ports, the device could have (i) a priming volume PV≈150 mL, (ii) an estimated pressure drop ΔP_{FR} of ≈9 mmHg for a nominal maximum blood flow rate F_{FR}=12*F_{RCC}≈4 L/min, and (iii) a total membrane surface area of SA_{FR}≈16*12*0.47 cm²≈0.9 m². The total reactor core primary fluid input area could be ≈12*16*66 cm²=112 cm². The fluid reactor could contain a heat exchanger 400 or 450 or electrical heater 500 with a surface area ~ 700 cm².

If the fluid channel diameter φ_{FC} and/or gap g_{FZ} are changed to get the lowest ΔP_{FR} with sufficient O₂/CO₂ gas transfer rate, the PAAL device can be further reduced in size and/or pressure drop ΔP_{FR}. In embodiments, any O₂/CO₂ gas transfer excess capacity can be reduced by designing a PAAL device having close to a minimal membrane surface area of SA_{FR} for the constraint of a total pressure drop per liter of blood flow ΔP_{FR}/F_{FR}≤3 mmHg/L or any other desirable ΔP_{FR}/F_{FR} ratio.

It should be understood that if the performance of actual parts is different from the above assumed nominal values, the concepts, and methods described herein can be applied to design a suitable device that is compatible with the actual gas transfer rate obtainable of the chosen rector core elements.

As discussed above, any oxygenator design options can further benefit from additional biocompatible coatings, such as antithrombotic coatings, coatings that reduce inflammatory side effects (particularly in the kidney), coatings that reduce coagulations, and/or coatings that reduce other possible blood damage. Such coatings can improve the quality of life of patients and/or extend the usability timeframe for such fluid reactors.

Based on the above teachings, those skilled in the art can optimize the herein discussed methods and design options for fluid reactors and their various parts for other fluid reactor applications and/or fluid reactor performance goals. Obvious extensions to fluid reactors, the manufacturing methods, and the herein discussed applications, as well obvious derivations, are intended to be included in this disclosure.

1. A reactor core component comprising:
a reactor core frame having a first window, a second window, and defining a secondary fluid cavity; and
at least one reactor core element having a primary fluid input surface, a primary fluid output surface, and a sidewall, the at least one reactor core element being secured to the reactor core frame via one or more fluid tight seals, at least one side of at least one of the first or second windows being larger in width or length than the corresponding width or length of the at least one reactor core element, wherein:
at least a portion of the primary fluid input surface is exposed through the first window, at least a portion of the primary fluid output surface is exposed through the second window and at least a portion of the reactor core element sidewall is positioned within the secondary fluid cavity; and
the primary fluid input surface includes a plurality of fluid channels extending through the at least one reactor core element to the primary fluid output surface, each channel of the plurality of fluid channels:
being surrounded by an open-pore cellular network material having a bi-continuous tortuous phase structure; and
defining a sidewall that allows a secondary fluid to pass through the sidewall of the channel while restricting the flow of at least one component of a primary fluid through the sidewall of the channel.

2. The reactor core component of claim 1 wherein the at least one reactor core element has a width smaller than its length.

3. The reactor core component of claim 2 wherein the at least one reactor core element has a width to length ratio from 1:3 to 1:12.

4. The reactor core component of claim 1, wherein the reactor core frame includes a first frame piece assembled with a second frame piece to form the reactor core frame, the first frame piece defining the first window and the second frame piece defining the second window.

5. The reactor core component of claim 4, further comprising turbulence generators spanning from the first frame piece to the second frame piece in the secondary fluid cavity.

6. The reactor core component of claim 1 wherein the reactor core frame includes a primary fluid input manifold sheet, an input frame sheet, an output frame sheet, a primary fluid output manifold sheet, and a pair of spacers between the input frame sheet and the output frame sheet.

7. The reactor core component of claim 1, wherein the at least one reactor core element is secured to the reactor core frame via a UV-curable glue.

8. The reactor core component of claim 1, wherein the at least one reactor core element is a reactor core stack including a plurality of reactor core elements.

9. The reactor core component of claim 1, wherein two or more of the reactor core elements of the plurality of reactor core elements are connected in parallel.

10. The reactor core component of claim 1, wherein two or more of the reactor core elements of the plurality of reactor core elements are connected in series.

11. The reactor core component of claim 1, wherein at least a portion of the open-pore cellular network material having a bi-continuous tortuous phase structure is coated.

12. The reactor core component of claim 11, wherein at least a portion of the open-pore cellular network material having a bi-continuous tortuous phase structure is coated with a hydrophobic coating.

13. The reactor core component of claim 12 wherein the hydrophobic coating is a hydrophobic polymer.

14. The reactor core component of claim 13 wherein the hydrophobic polymer is a fluorocarbon material.

15. A reactor core component comprising:
a reactor core frame including a first frame piece assembled with a second frame piece, the reactor core frame having a plurality of pairs of windows, each pair of windows of the plurality of pairs of windows defined by a first window in the first frame piece, and a second window in the second frame piece, the reactor core frame defining a secondary fluid cavity; and
a plurality of reactor core stacks each having a primary fluid input surface, a primary fluid output surface, and a sidewall, each reactor core stack of the plurality of reactor core stacks being secured to the reactor core frame between a respective pair of windows of the plurality of pairs of windows via one or more fluid tight seals, at least one side of at least one of the first or second windows of each pair of windows of the plurality of pairs of windows being larger in width or length than the corresponding width or length of the respective reactor core stack secured therein, wherein for each reactor core stack of the plurality of reactor core stacks:
at least a portion of the primary fluid input surface is exposed through the first window, at least a portion of the primary fluid output surface is exposed through the second window, and at least a portion of the reactor core stack sidewall is positioned within the secondary fluid cavity; and
the primary fluid input surface includes a plurality of fluid channels extending through the reactor core stack to the primary fluid output surface, each channel of the plurality of fluid channels:
being surrounded by an open-pore cellular network material having a bi-continuous tortuous phase structure; and
defining a sidewall that allows a secondary fluid to pass through the sidewall of the channel while restricting the flow of at least one component of a primary fluid through the sidewall of the channel.

16. The reactor core component of claim 15, wherein at least one reactor core stack of the plurality of reactor core stacks includes two or more reactor core elements.

17. The reactor core component of claim 16, wherein the two or more of the reactor core elements are connected in parallel.

18. The reactor core component of claim 16, wherein the two or more of the reactor core elements are connected in series.

19. A reactor core assembly comprising:
a first reactor core component in accordance with claim 15; and
at least one of:
a second reactor core component in accordance with claim 15; or
a reactor core accessory selected from a heat exchanger or a sensor.

20. A fluid reactor unit comprising:
a reactor core component in accordance with claim 15 sealed within a fluid reactor unit housing including a first endplate and a second endplate, the housing including a primary fluid input port, a primary fluid output port, and at least one of a secondary fluid input port or a secondary fluid output port.

21. A fluid reactor unit comprising:
at least one reactor core component in accordance with claim 15 sealed within a housing including a lid and a container, the housing having and a primary fluid input port, a primary fluid output port, and at least one of a secondary fluid input port or a secondary fluid output port.

22. A fluid reactor unit comprising:
a first plurality of reactor core assembly parts, the first plurality of reactor core assembly parts including at least one of a reactor core component in accordance with claim 15 and optionally a reactor core accessory;
a second plurality of reactor core assembly parts, the second plurality of reactor core assembly parts including at least one of a reactor core component in accordance with claim 15 or a reactor core accessory;
the first and second pluralities of reactor core assembly parts sealed within a housing including a lid and a container, the housing having a primary fluid input port, a primary fluid output port, and at least one of a secondary fluid input port or a secondary fluid output port.

23. A fluid reactor unit comprising:
a plurality of reactor core assembly parts sealed within a housing, the plurality of reactor core assembly parts including at least two reactor core components in accordance with claim 15, the at least two reactor core components positioned adjacent to one another such that a window of one of the at least two reactor core components is serially connected with a window of another of the at least two reactor core components.

24. A method of making a fluid reactor for compositionally changing a primary input fluid into a primary output fluid with at least one secondary fluid comprising:
securing a reactor core component within a fluid tight fluid reactor housing, the reactor core component being prepared by:
loading a reactor core element into a window of a reactor core frame, where the reactor core frame has a secondary fluid cavity, the reactor core element having:
a sidewall and a plurality of fluid channels surrounded by an open-pore cellular network material having a bi-continuous tortuous phase structure that is configured to be transmissive to a secondary fluid, and
at least one side of the window is larger in width and length than the reactor core element; and
bonding the reactor core element to the window such that at least a portion of the reactor core element sidewall is not sealed.

25. The method of claim 24 wherein loading a reactor core element into a window of a reactor core frame includes loading a reactor core stack into a window of a reactor core frame.

26. The method of claim 24 wherein the reactor core frame includes a plurality of windows, and the method includes loading each reactor core element of a plurality of reactor core elements into a respective window of the plurality of windows.

27. The method of claim 26 wherein loading each reactor core element of a plurality of reactor core elements into a respective window of the plurality of windows includes loading at least one reactor core stack into a window of a reactor core frame.

28. The method of claim 27 wherein loading at least one reactor core stack into a window of a reactor core frame includes loading at least one reactor core stack that includes two or more reactor core elements.

29. The method of claim 28 wherein loading at least one reactor core stack into a window of a reactor core frame includes loading at least one reactor core stack that includes two or more reactor core elements that are connected in parallel.

30. The method of claim 28 wherein loading at least one reactor core stack into a window of a reactor core frame includes loading at least one reactor core stack that includes two or more reactor core elements that are connected in series

31. The method of claim 24 wherein two or more reactor core components are secured within the fluid tight fluid reactor housing.

32. The method of claim 24 further comprising securing at least one reactor core accessory within the fluid tight fluid reactor housing.

33. The method of claim 32 wherein the at least one reactor core accessory is a heat exchanger.

34. A method of compositionally transforming a primary fluid, the method comprising:
flowing a primary input fluid through at least one first input port of a fluid reactor and into and through a primary fluid input manifold, where the primary fluid input manifold includes at least one primary fluid line and at least one primary fluid input cavity;
flowing the primary input fluid over a reactor core component containing at least one reactor core element located in fluid communication with the primary fluid cavity such that the primary input fluid enters at least one fluid channel located within the at least one reactor core element;
flowing a secondary input fluid through at least one second input port of the fluid reactor and into and through a secondary fluid input manifold, where the secondary fluid input manifold includes at least one secondary fluid line and at least a portion of at least one secondary fluid cavity;
guiding the secondary input fluid from the secondary fluid cavity across the at least one reactor core element in a manner perpendicular to a direction of flow of the primary input fluid;
interacting the primary input fluid with the secondary input fluid whereby the primary input fluid is compositionally changed into a primary output fluid and the secondary input fluid is compositionally changed into a secondary output fluid;
flowing a primary output fluid out of the at least one fluid channel and into and through a primary fluid output manifold, where the primary fluid output manifold includes at least one primary fluid line and at least one primary fluid output cavity;
flowing the primary output fluid from the primary fluid output manifold through at least one primary fluid output port from where it exists the fluid reactor;
flowing a secondary output fluid out of the at least one reactor core element and into and through a secondary fluid output manifold, where the secondary fluid output manifold includes at least one secondary fluid line and at least a portion of at least one secondary fluid output cavity; and
flowing the secondary output fluid from the secondary fluid output manifold through at least one secondary fluid output port from where it exits the fluid reactor.

35. The method of claim 34 wherein the primary input fluid is blood, brackish water, salt water, water, or an industrial fluid.
